(19) United States
(12) Patent Application Publication
(10) Pub. No.: US 2021/0016284 A1
(43) Pub. Date: Jan. 21, 2021

(54) MICROFLUIDIC DEVICE

(71) Applicant: Heriot-Watt University, Edinburgh (GB)

(72) Inventors: Maiwenn KERSAUDY-KERHOAS, Edinburgh (GB); Witold MIELCZAREK, Edinburgh (GB); Antonio LIGA, Edinburgh (GB)

(21) Appl. No.: 17/042,585

(22) PCT Filed: Mar. 29, 2019
(86) PCT No.: PCT/GB2019/050935

§ 371 Date: Sep. 28, 2020

(30) Foreign Application Priority Data
Mar. 29, 2018 (GB) 1805145.8

(51) Int. Cl. B01L 3/00; B01D 17/02; G01N 33/49

(52) U.S. Cl. CPC B01L 3/502753; B01L 3/502746; B01D 17/0214; G01N 33/491; B01L 2400/0487; B01L 2200/0631; B01L 2200/0652; B01L 2200/12; B01L 2300/0864; B01L 2200/0621

(57) ABSTRACT

A microfluidic device (200) for separating a liquid L into first and second liquid components L₁, L₂ thereof is described. The microfluidic device (200) comprises an inlet (230) for receiving the liquid therethrough. The microfluidic device (200) comprises a first outlet (210) for the first liquid component L₁, wherein the first outlet (210) is fluidically coupled to the inlet (230) via a first passageway (240). The microfluidic device (200) comprises a second outlet (220) for the second liquid component L₂, wherein the second outlet (220) is fluidically coupled to the first passageway (240A) via a first set of N conduits 250 (250A, 250B, 250C, 250D, 250E), wherein N is a positive integer greater than 1, wherein respective conduits 250A, 250B, 250C, 250D, 250E of the first set of N conduits 250 divide from the first passageway 240A at respective divisions 252 (252A, 252B, 252C, 252D, 252E) from the inlet 230 therealong 240. The respective conduits 250A, 250B, 250C, 250D, 250E of the first set of N conduits 250 are arranged to, at least in part, equalize flowrate ratios at the respective divisions 252 (252A, 252B, 252C, 252D, 252E).

### FIELD

The present invention relates to microfluidic devices for separating liquids. Particularly, the present invention relates to microfluidic devices for separating liquids into different liquid components, for example for separating plasma from whole blood.

### BACKGROUND TO THE INVENTION

Conventional blood plasma separation techniques, for example centrifugation, provide high throughputs, high plasma yields and high plasma purities, while reducing cellular damage and thereby preventing contamination of separated plasma with cellular DNA and haemoglobin. However, these conventional blood plasma separation techniques are not suitable for lab-on-a-chip devices, due to at least requirements for reduction in scale and/or cost and/or a transition from offline (also known as non-continuous) processing to inline (also known as continuous processing). Blood plasma separation techniques proposed for such lab-on-a-chip devices include miniaturised centrifugation, miniaturised filtration and microfluidic separation. However, miniaturised centrifugation is generally an offline processing technique that is not compatible with inline processing requirements of the lab-on-a-chip devices. Furthermore, miniaturised filtration is susceptible to blockages, thereby reducing robustness of the lab-on-a-chip devices. In addition, known microfluidic separation techniques provide relatively low throughputs.

Hence, there is a need to improve separation of liquids, for example for separating biological fluids such as for separating plasma from whole blood on lab-on-a-chip devices.

### SUMMARY OF THE INVENTION

It is one aim of the present invention, amongst others, to provide a microfluidic device which at least partially obviates or mitigates at least some of the disadvantages of the prior art, whether identified herein or elsewhere. For instance, it is an aim of embodiments of the invention to provide a microfluidic device for separating a liquid into first and second liquid components thereof at an improved throughput, such as at a higher flowrate and/or a higher pressure of the liquid. For instance, it is an aim of embodiments of the invention to provide a microfluidic device for separating a liquid into first and second liquid components thereof that provides improved yields and/or purities of the separated first and/or second liquid components. For instance, it is an aim of embodiments of the invention to provide a microfluidic device for separating a liquid into first and second liquid components thereof that is suitable for inline processing. For instance, it is an aim of embodiments of the invention to provide a microfluidic device for separating a liquid into first and second liquid components thereof that is suitable for lab-on-a-chip devices. For instance, it is an aim of embodiments of the invention to provide a microfluidic device for separating biological fluids, for example whole blood into plasma and waste.

A first aspect provides a microfluidic device for separating a liquid into first and second liquid components thereof, the microfluidic device comprising:

an inlet for receiving the liquid therethrough;

a first outlet for the first liquid component, wherein the first outlet is fluidically coupled to the inlet via a first passageway; and

a second outlet for the second liquid component, wherein the second outlet is fluidically coupled to the first passageway via a first set of N conduits, wherein N is a positive integer greater than 1, wherein respective conduits of the first set of N conduits divide from the first passageway at respective divisions from the inlet therealong;

wherein the respective conduits of the first set of N conduits are arranged to, at least in part, equalize flowrate ratios at the respective divisions.

A second aspect provides an apparatus arranged to control a microfluidic device according to the first aspect.

A third aspect provides a microfluidic system comprising an apparatus according to the second aspect and a microfluidic device according to the third aspect.

A fourth aspect provides a method of operating a microfluidic system according to the third aspect.

A fifth aspect provides a lab-on-a-chip device comprising a microfluidic device according to the first aspect.

### DETAILED DESCRIPTION OF THE INVENTION

According to the present invention there is provided a microfluidic device, as set forth in the appended claims. Also provided is an apparatus arranged to control such a microfluidic device, a microfluidic system and a method of operating such a microfluidic system. Other features of the invention will be apparent from the dependent claims, and the description that follows.

Throughout this specification, the term “comprising” or “comprises” means including the component(s) specified but not to the exclusion of the presence of other components. The term “consisting essentially of” or “consists essentially of” means including the components specified but excluding other components except for materials present as impurities, unavoidable materials present as a result of processes used to provide the components, and components added for a purpose other than achieving the technical effect of the invention, such as colourants, and the like.

The term “consisting of” or “consists of” means including the components specified but excluding other components.

Whenever appropriate, depending upon the context, the use of the term “comprises” or “comprising” may also be taken to include the meaning “consists essentially of” or “consisting essentially of”, and also may also be taken to include the meaning “consists of” or “consisting of”.

The optional features set out herein may be used either individually or in combination with each other where appropriate and particularly in the combinations as set out in the accompanying claims. The optional features for each aspect or exemplary embodiment of the invention, as set out herein are also applicable to all other aspects or exemplary embodiments of the invention, where appropriate. In other words, the skilled person reading this specification should consider the optional features for each aspect or exemplary embodiment of the invention as interchangeable and combinable between different aspects and exemplary embodiments.

Microfluidic Device

The first aspect provides a microfluidic device for separating a liquid into first and second liquid components thereof, the microfluidic device comprising:

an inlet for receiving the liquid therethrough;

a first outlet for the first liquid component, wherein the first outlet is fluidically coupled to the inlet via a first passageway; and

a second outlet for the second liquid component, wherein the second outlet is fluidically coupled to the first passageway via a first set of N conduits, wherein N is a positive integer greater than 1, wherein respective conduits of the first set of N conduits divide from the first passageway at respective divisions from the inlet therealong; wherein the respective conduits of the first set of N conduits are arranged to, at least in part, equalize flowrate ratios at the respective divisions.

In this way, the microfluidic device may separate the liquid into the first and second liquid components thereof at an improved throughput, such as at a higher flowrate and/or a higher pressure of the liquid. In this way, the microfluidic device may separate the liquid into the first and second liquid components thereof at improved yields and/or purities of the separated first and second liquid components. In this way, the microfluidic device may separate the liquid into the first and second liquid components thereof inline and thus suitable for inline processing. In this way, the microfluidic device may separate the liquid into the first and second liquid components thereof at a scale and/or cost that is suitable for lab-on-a-chip devices. In this way, the microfluidic device may separate whole blood into plasma and waste. Particularly, by equalizing flowrate ratios at the respective divisions, efficiencies of separation at each respective division may be improved, even at higher flowrates of the liquid. In turn, this may result in improved yields and/or purities of the separated first and/or second liquid components. For example, the microfluidic device may be used to extract liquid phase (i.e. the second liquid component) from a suspension (i.e. the liquid).

It should be understood that the microfluidic device is a microfluidic device. Microfluidics typically relates to behaviour, control and/or manipulation of fluids that are geometrically constrained to small, typically sub-millimeter, scales, such as microscales from about 100 nm to about 500 μm. Microfluidic behaviour may differ from macrofluidic behaviour since effects due to surface tension, energy dissipation and/or fluidic resistance, which may be negligible in macrofluidics, may instead tend to predominate in microfluidics. For example, the Reynolds number of the fluid may decrease significantly at the microscale. Generally, the Reynolds number is a ratio of inertial forces to viscous forces within a fluid which is subjected to relative internal movement due to different fluid velocities, in which is known as a boundary layer in the case of a bounding surface such as the interior of a pipe. At the microscale, the viscous forces dominate and the inertial forces may be negligible. Thus, flow of the fluid may be laminar at the microscale, rather than turbulent as at the macroscale. Hence, co-flowing fluids, for example co-flowing first and second fluid components, in continuous-flow microfluidics may not mix effectively at the microscale, due to this laminar, rather than turbulent, flow. Instead, mixing of the co-flowing fluids may be by diffusional molecular transport. Such diffusional mixing may tend to reduce mixing efficiency while increasing mixing timescales, by up to orders of magnitude. Furthermore, at the microscale, a mass transfer Peclet number may be large, affecting microfluidic mixing. Generally, the mass transfer Peclet number is the product of the Reynolds number and the Schmidt number, the latter defined as the ratio of momentum diffusivity (kinematic viscosity) to mass diffusivity. Conversely, mixed first and second fluid components in continuous-flow microfluidics may not separate effectively at the microscale, thereby reducing separation performance, particularly at higher flowrates. The separation performance may be defined in terms of a yield and/or a purity. For example, for separation of blood (also known as fractionation) in which the second liquid component is ideally pure plasma, the yield may be defined as the ratio of the amount of collected plasma (i.e. the second liquid component) to the total amount of plasma in the blood (i.e. the liquid). The yield may be expressed as a percentage. Additionally and/or alternatively, the purity may be defined based on a red blood cell count, specifically one minus the ratio of the number of red blood cells in the collected plasma (i.e. the second liquid component) to the total number of red blood cells in the blood (i.e. the liquid). The purity may be expressed as a percentage. In addition, microfluidics may often involve particles, for example transport thereof, having sizes, for example diameters, in a range from about 10 nm to about 50 μm. Such particles may further modify microfluidic flow, mixing and/or separation.

Fluid Flow

Generally, fluid flows may be determined according to the Navier-Stokes equations, which consider gravitational, pressure and viscous forces. However, for microfluidics, certain assumptions may be made which may simplify microfluidic flow calculations. For example, microfluidic liquid flows may be unidirectional, gravitational effects may be neglected, convective terms may be neglected, the liquid may be incompressible, the liquid may be

Newtonian and/or the Reynolds number may be small, such that the relevant Navier-Stokes equation approximates to the Stokes equation in which viscous forces balance pressure forces.

$$ρ \frac{δ}{δ} = - ∇ p + μ {∇}^{2} + f$$

where ρ is the density of the liquid,

$$\frac{δ}{δ}$$

is the liquid velocity and μ is the viscosity of the liquid and the other terms have their usual meanings.

Pressure-driven Microflow

The Navier-Stokes equation may be solved for various shapes of microchannels. In the case of cylindrical microchannels, a parabolic flow develops and the relation between pressure and flow rate is described by the Hagen-Poiseuille equation:

$$Δ P = \frac{8 η}{π {r}^{4}} = \frac{8 η}{{r}^{2}} = \frac{32 η}{{D}^{2}}$$

where ΔP is the pressure drop between the two ends of the channel, L is the total length of channel, Q is the volumetric flow rate, r is the radius of the channel (or D the diameter) and υ is the average flow velocity across the section.

The relation between pressure and flow rate may be similarly determined or approximated for other shapes of microchannels, for example square microchannels and/or low aspect ratio rectangular microchannels.

Liquid

The microfluidic device is for separating the liquid into the first and second liquid components thereof. That is, the liquid comprises a mixture of the first and second liquid components and the microfluidic device may be used to separate, for example at least partially separate or fully separate, the mixture.

In one example, the liquid comprises and/or is an emulsion wherein the first liquid component (also known as a dispersed phase) is dispersed in the second liquid component (also known as a continuous phase) or vice versa, whereby the microfluidic device may be used to separate, for example at least partially separate or fully separate, the first and second liquid components therefrom.

In one example, the liquid comprises and/or is a suspension. In one example, the liquid comprises and/or is a colloid (also known as a colloidal suspension) comprising dispersed-phase particles whereby the microfluidic device may be used to separate, for example at least partially separate or fully separate, the first and second liquid components therefrom having respectively higher and lower concentrations of the dispersed-phase particles, or vice versa. In this way, the first liquid component may be relatively enriched with respect to the dispersed-phase particles while the second liquid component may be relatively depleted with respect to the dispersed-phase particles, or vice versa. In this way, the dispersed-phase particles may be extracted from the liquid by concentration in the first liquid component, for example. Conversely, the second liquid component may be provided having a relatively lower concentration of the dispersed phase particles, for example, being substantially free therefrom. In one example, the dispersed-phase particles have a diameter or characteristic dimension in a range from 1 nm to 10 μm, preferably in a range from 100 nm to 5 μm, more preferably in a range from 1 μm to 3 μm.

In one example, the liquid comprises and/or is a biological fluid, for example blood (also known as whole blood). In one example, the liquid is blood (also known as whole blood). Dispersed-phase particles in whole blood include leukocytes (white blood cells), platelets and/or erythrocytes (red blood cells). Typically, fractionation of whole blood by centrifugation results in three components: a clear solution of blood plasma; a buffy coat, which is a thin layer of leukocytes mixed with platelets; and erythrocytes. In one example, the first liquid component comprises leukocytes, platelets and/or erythrocytes. In one example, the second liquid component comprises and/or is separated blood plasma. In one example, the second liquid component comprises and/or is separated blood plasma, relatively free from leukocytes, platelets and/or erythrocytes, for example comprising at most 10%, at most 5%, at most 3%, at most 2%, at most 1%, at most 0.5% or at most 0.1% leukocytes, platelets and/or erythrocytes by mass. In this way, the whole blood may be fractionated. In fractionation of whole blood, such a second liquid component, being substantially blood plasma, may be used for testing while such a first liquid component may be known as waste. In one example, the liquid is whole blood. In one example, the liquid is diluted whole blood, for example whole blood diluted by a ratio of at most 10:1, preferably at most 5:1 more preferably at most 2:1, most preferably at most 1:1.

Inlet

The microfluidic device comprises the inlet for receiving the liquid therethrough. In this way, the liquid may be admitted into the microfluidic device, for example by pumping using a pump such as a syringe pump or a peristaltic pump. In one example, the inlet comprises a fluidic coupling for coupling a pipe, tube or capillary thereto, such as a pushfit coupling, a quick release coupling, a bayonet coupling or a compression threaded coupling. In one example, the microfluidic device comprises a single (i.e. only one) inlet for the liquid. In this way, a number of fluidic couplings or connections to be made for use of the microfluidic device may be reduced, reducing cost, complexity and/or risk of leakage.

First Outlet

The microfluidic device comprises the first outlet for the first liquid component. In this way, the first liquid component may be exhausted or discharged from the microfluidic device via the first outlet. In one example, the first outlet comprises a fluidic coupling, as described with respect to the inlet. In one example, the microfluidic device comprises a single (i.e. only one) first outlet for the first liquid component. In this way, a number of fluidic couplings or connections to be made for use of the microfluidic device may be reduced, reducing cost, complexity and/or risk of leakage.

The first outlet is fluidically coupled to the inlet via the first passageway. In other words, the and first outlet is in fluid communication with the inlet via the first passageway. In this way, at least a part of the liquid (i.e. the first liquid component) received through the inlet may flow, in use, from the inlet to the outlet and therethrough. In one example, the first passageway directly connects the inlet to the first outlet.

First Passageway

In one example, a cross-sectional dimension, for example a width, a height, a diameter and/or a cross-sectional area, of the first passageway is relatively large, compared with a conduit of the first set of N conduits, as described below. In this way, a backpressure due to flow of the liquid therethrough is reduced. In one example, a width, a height and/or a diameter of the first passageway is in a range from 50 μm to 500 μm, preferably in a range from 75 μm to 250 μm, more preferably in a range from 90 μm to 150 μm, for example 100 μm. In one example, a cross-sectional area of the first passageway is in a range from 2500 μm² to 250000 μm², preferably in a range from 5625 μm² to 62500 μm², more preferably in a range from 8100 μm² to 22500² μm, for example 10000 μm².

In one example, a cross-sectional shape of the first passageway is a symmetrical shape, for example a symmetric oval, a circle, a symmetric polygon such as a square or rectangle. In this way, flow characteristics of the liquid therein may be better controlled while a complexity and/or cost may be reduced. In one example, a cross-sectional shape of the first passageway is constant along at least a part of the length thereof, preferably along substantially the length thereof, for example along at least 50%, at least 60%, at least 70%, at least 80%, at least 90%, at least 95% or at least 97.5% of the length thereof. In this way, flow characteristics of the liquid therein may be better controlled while a complexity and/or cost may be reduced. In one example, internal corners of the first passageway are smooth and/or radiused (also known as filleted). In this way, flow of the liquid therein may be more uniform, thereby reducing mixing of the liquid. In one example, the first passageway includes no internal corners. In one example, internal surfaces of the first passageway are smooth, having no protuberances or recesses. In this way, flow of the liquid therein may be more uniform, thereby reducing mixing of the liquid, and/or reducing unswept dead volumes.

In one example, a length of the first passageway is relatively long. In this way, stability of flow of the liquid therein may be improved. In one example, a length of the first passageway is in a range from 1 to 100 mm, preferably in a range from 10 to 80 mm, more preferably in a range from 20 to 60 mm. In one example, an aspect ratio (i.e. a ratio of a length to a cross-section dimension such as width, height or diameter) of the first passageway is in a range from 20 to 2000, preferably in a range from 50 to 1500, more preferably in a range from 100 to 1000.

Taper

In one example, the first passageway tapers from the inlet towards the first outlet along at least a part of a length thereof. Formation of vortices, for example, which promote mixing of the liquid and hence the first liquid component and the second liquid component, is undesired, being contrary to a purpose of the device. Hence, the formation of vortices should be reduced and/or prevented. By tapering the first passageway, for example, from the inlet towards the first outlet along at least a part of a length thereof, for example from a first larger cross-sectional area to a second smaller cross-sectional area, the formation of vortices may be reduced and/or prevented while a stability of flow of the liquid may be improved.

In one example, a cross-sectional dimension, for example a width, a height, a diameter and/or a cross-sectional area, of the first passageway is constant along a length thereof. In one example, the first passageway tapers, for example uniformly, along a part a length thereof. In this way, stability of flow of the liquid therethrough may be improved. In one example, the first passageway tapers, for example uniformly, along a part a length thereof, wherein in a reduction in a cross-sectional dimension, for example a width, a height, a diameter and/or a cross-sectional area, is in a range from 10% to 90%, preferably in a range from 20% to 75%, more preferably in a range from 30% to 60%. In one example, the first passageway tapers, for example uniformly, along a part of the length thereof in a range from 5% to 90%, preferably in a range from 10% to 75%, more preferably in a range from 20% to 50% of the length. In one example, the first passageway tapers from and/or proximal from the inlet towards the first outlet. In one example, a cross-sectional dimension, for example a width, a height, a diameter and/or a cross-sectional area, of the first passageway along a remaining length (i.e. excluding the part of the length along which the first passageway tapers) is constant.

Radius of Curvature

In one example, the first passageway curves (i.e. has an arcuate form) from the inlet towards the first outlet along a first part of a length thereof, wherein a first radius of curvature of the first part is in a range from 1 mm to 500 mm, preferably in a range from 5 mm to 50 mm. In one example, the first passageway curves (i.e. has an arcuate form) from the inlet towards the first outlet along a second part of a length thereof, wherein a second radius of curvature of the second part is in a range from 1 mm to 500 mm, preferably in a range from 5 mm to 50 mm. In one example, the first passageway tapers along the first part of the length. In one example, the first passageway has a constant cross-sectional area along the second part of the length. In one example, the first passageway is linear along a third part of a length thereof, between the first part of the length and the second part of the length. In one example, the first passageway has a constant cross-sectional area along the third part of the length.

Such relatively large radii of curvature (i.e. the first radius of curvature and/or the second radius of curvature) may reduce an effect due, at least in part, to high shear forces that may otherwise disrupt flow of the liquid. In contrast, relatively smaller radii of curvature may introduce high shear forces, which may disrupt flow of the liquid, for example flow of blood cells in blood. For example, a serpentine first passageway having relatively smaller radii of curvature may disrupt the formation of a cell-free layer in blood while also having a relatively large footprint, for example occupying real-estate on a blood separation chip. Particularly, the presence of tight bends in a serpentine first passageway may destabilise a cell-free layer, as the centrifugal force tends to widen an inner cell-free layer and reduce an outer layer at the exit of each tight bend, creating a fluctuation of the cell-free layer width around its mean value. In one example, the first radius of curvature and the second radius of curvature are in a same direction, for example clockwise or counter-clockwise. By curving only in one direction, a second liquid component layer remains relatively stable while the centrifugal force tends to widen an inner layer of the second liquid component and reduce an outer layer thereof at the exit of the relatively large radii of curvature, which then corresponds with the effect due to the set of the set of constriction members are arranged proximal to and/or on a same side. For example, during separation of blood, by curving only in one direction, the cell-free layer remains relatively stable while the centrifugal force tends to widen the inner cell-free layer and reduce the outer layer at the exit of the relatively large radii of curvature. In the separation of blood, for example, a lift force is thus imparted on cells in one direction.

Surround

In one example, the first passageway is arranged to surround, at least in part, the first set of N conduits. That is, the first passageway may be arranged around at least a part of a periphery of the first set of N conduits. In this way, a footprint of the microfluidic device may be reduced.

In one example, the first passageway curves from the inlet towards the first outlet along a first part of a length thereof, wherein a first radius of curvature of the first part is in a range from 1 mm to 500 mm, preferably in a range from 5 mm to 50 mm, and wherein the first passageway tapers along the first part of the length, the first passageway curves from the inlet towards the first outlet along a second part of a length thereof, wherein a second radius of curvature of the second part is in a range from 1 mm to 500 mm, preferably in a range from 5 mm to 50 mm, and wherein the first passageway has a constant cross-sectional area along the second part of the length, the first passageway is linear along a third part of a length thereof, between the first part of the length and the second part of the length wherein the first passageway has a constant cross-sectional area along the third part of the length and wherein the first passageway is arranged to surround, at least in part, the first set of N conduits.

Linear Flow Path

In one example, the first passageway defines a linear flow path of the liquid via the respective divisions. In this way, stability of flow of the liquid, for example reformation of a cell-free layer therein, after each division may be improved. In one example, the first passageway defines a linear flow path of the liquid via the respective divisions, wherein a wall, for example opposed to the first set of N conduits, of the first passageway extending between the respective divisions is linear.

Non-linear Flow Path

In one example, the first passageway defines a non-linear flow path, for example a smoothly curved flow path, of the liquid via the respective divisions. In this way, stability of flow of the liquid, for example reformation of a cell-free layer therein, after each division may be improved.

In one example, the first passageway defines a non-linear flow path of the liquid via the respective divisions, wherein a wall, for example opposed to the first set of N conduits, of the first passageway extending between the respective divisions is non-linear.

Second Outlet

The microfluidic device comprises the second outlet for the second liquid component. In this way, the second liquid component may be exhausted or discharged from the microfluidic device via the second outlet. In one example, the second outlet comprises a fluidic coupling, as described with respect to the inlet and/or the first outlet. In one example, the microfluidic device comprises a single (i.e. only one) second outlet for the second liquid component. In this way, a number of fluidic couplings or connections to be made for use of the microfluidic device may be reduced, reducing cost, complexity and/or risk of leakage.

Conduits

The second outlet is fluidically coupled to the first passageway via the first set of N conduits (also known as channels, passageways, capillaries, tubes or pipes). That is, the first passageway is divided, for example bifurcated, by the respective conduits of the first set of N conduits and the respective conduits of the first set of N conduits are in fluid communication with the second outlet. In other words, each conduit branches or divides from the first passageway. In this way, respective conduits from the first set of N conduits may provide a Zweifach-Fung bifurcation effect, as described herein, whereby the first liquid component tends to continue to flow therealong at the respective divisions and the second liquid component tends to preferentially flow into the respective conduits at the respective divisions.

In this way, the liquid may be separated by these divisions into the first liquid component and the second liquid component by the microfluidic device such that the first liquid component tends to flow out of (i.e. discharged from) the microfluidic device via the first outlet and the second liquid component tends to flow out of (i.e. discharged from) the microfluidic device via the second outlet. In one example, respective conduits of the first set of N conduits bifurcate the first passageway. In one example, the first passageway is bifurcated by the respective conduits of the first set of N conduits.

The respective conduits of the first set of N conduits divide from the first passageway at respective divisions from the inlet therealong. That is, the first passageway may be bifurcated by the respective conduits of the first set of N conduits such that respective divisions of the first passageway thus defined are mutually spaced apart. In other words, each conduit branches or divides from the first passageway at a different position therealong. In one example, the respective conduits of the first set of N conduits divide from the first passageway at respective divisions from the inlet therealong, wherein the respective divisions between adjacent conduits are equal.

Fluidic Resistance

The average flow rate Q of a liquid within a micro- or nanofluidic channel is proportional to the pressure gradient ΔP imposed on both ends of the capillary. As a consequence, the Hagen-Poiseuille equation can be rewritten as a classical Ohm's law type equation for electrical resistance:

ΔP=R_{f}Q

The fluidic resistance R_{f} will depend on the geometry of the cross section.

The fluidic resistance can also be calculated for micro- and nanofluidic networks using the same method as for electrical circuits and the flow rates can be deduced in the different portions of the microfluidic device, as an example using the classical Kirchhoff equations. This concept can be advantageously used in microfluidics by using capillary tubing that will act as flow restrictors and let the user reach and work with low flow rates, even with a low fluidic resistance setup.

Applying the same method as for electrical circuits, thereby considering fluidic resistances parts of the first passageway and respective fluidic resistances of the set of N conduits for the microfluidic device, equations may be defined to impose flow rate ratios, flow rate conservation and the mesh rule. For example, for N=2 (i.e. two conduits hence two divisions, specifically bifurcations, effectively defining three parts of the first passageway between the inlet and the first outlet):

Flowrate ratios:

q₁=η₁Q₁

q₂=η₂Q₂

Flow rate conservation:

Q₁=Q₂+q₁

Q₂=Q₃+q₂

Mesh rule:

r₁q₁=R₂Q₂+r₂q₂

r₂q₂=R₃Q₃

Where Q₁ is the flowrate in the first passageway prior to the first division (i.e. the inlet flowrate), Q₂ is the flowrate in the first passageway between the first division and the second division, Q₃ is the flowrate in the first passageway after the second division (i.e. the first outlet flowrate), q₁ is the flowrate in the first conduit, q₂ is the flowrate in the second conduit, η₁ is the volumetric extraction ratio due to the first division, η₂ is the volumetric extraction ratio due to the second division, R₁ is the fluidic resistance due to the first passageway prior to the first division, R₂ is the fluidic resistance due to the first passageway between the first division and the second division, R₃ is the fluidic resistance due to the first passageway after the second division, r₁is the fluidic resistance due to the first conduit and r₂ is the fluidic resistance due to the second conduit.

Hence:

$${r}_{1} = \frac{1 - {η}_{1}}{{η}_{1}} [ {R}_{2} + ( 1 - {η}_{2} ) {R}_{3} ]$$

$${r}_{2} = \frac{1 - {η}_{2}}{{η}_{2}} {R}_{3}$$

An overall extraction ratio η may be given by:

η=1−[(1−η₁)·(1−η₂)]

It should be understood that this method may be similarly applied to other values for N and/or for microfluidic devices comprising a second passageway and a second set of M conduits, as described below.

The respective conduits of the first set of N conduits have relatively high fluidic resistances (for example r₁ and r₂) due to their geometrical features (length and width), while the first passageway has relatively lower fluidic resistances (for example R₁, R₂ and R₃) due to having a relatively larger cross-sectional area. This difference in resistance at each division is proportional to the flow rate ratio at each division. Furthermore, the fluidic resistance of the first passageway decreases after each division. The flow rate ratios may be estimated using an algorithm implemented in mathematical modelling software such as MATLAB (Mathworks, Natick, USA).

Effective Section Calculation

The effective section Sₑ may be used to calculate the typical pressure drop as a function of the flow rate in microchannels. For complex micro- or nanofluidics networks, or when viscous liquids are used, the effective section can also be derived from the fluidic resistance calculation. Using the classical rules exposed here before, it is possible to get a first approximation of the global effective section of the microfluidic device using the total fluidic resistance Rₜ and the following equation:

$${S}_{e} \approx \sqrt{\frac{8 η}{π {R}_{t , ta}}}$$

In one example, the first passageway comprises and/or is a microfluidic first passageway. In one example, the respective conduits of the first set of N conduits comprise and/or are respective microfluidic conduits.

N Conduits

The first set of N conduits comprises and/or consists of the N conduits, wherein N is a positive integer greater than 1. In one example, N is in a range from 2 to 100, preferably in a range from 2 to 50, more preferably in a range from 3 to 10, for example 3, 4, 5, 6, 7, 8, 9 or 10. Particularly, by increasing N, while separation of the first liquid component and the second liquid component at each division may be relatively low, an overall separation efficiency is improved. For convenience, that conduit of the first set of N conduits closest to the inlet is referred to herein as the first conduit and successive conduits are referred to herein successively i.e. first conduit, second conduit, third conduit . . . Nth conduit (also known as the last conduit). Respective divisions are referred to herein similarly.

Divisions

The respective conduits of the first set of N conduits divide from the first passageway at respective divisions (also known as branching or forkings) from the inlet therealong i.e. along the first passageway. That is, a division is a branching or forking of the first passageway into a conduit of the first set of N conduits and a continuation thereafter of the first passageway. Separation of the liquid into the first liquid component and the second liquid component thereof may be due, at least in part, to the respective divisions, as described below in more detail in relation to a bifurcation law. In one example, the respective divisions are respective bifurcations. That is, the first passageway divides into 2 at the respective bifurcations: a respective conduit and the continuation of the first passageway. In one example, the first passageway divides into more than 2 at a division, for example into 3, 4 or more. For example, the first passageway may divide into 2, 3 or more conduits and the continuation of the first passageway at the division.

It should be understood that separation of the second liquid component from the liquid at the successive divisions (i.e. at successive conduits of the first set of N conduits) results in relative depletion of the second liquid component in the liquid that continues to flow therealong, such that the remaining liquid flowing in the first passageway after the last division is substantially the first liquid component, substantially free from the second liquid component. That is, a composition of the liquid flowing through the first passageway changes at successive divisions.

For convenience and without limitation, that liquid flowing through the first passageway is referred to herein as the liquid, being referred to as the first liquid component at the first outlet, having a composition finally determined by the last division. Similarly, compositions of the second liquid component in the respective conduits may be different due, at least in part to successive changes in the composition of the liquid at successive divisions. For example, a relative proportion of the first liquid component included with the second liquid component may be relatively higher in the first conduit than in the last conduit, or vice versa. In one example, the second liquid component in the respective conduits comprises at most 10%, at most 5%, at most 3%, at most 2%, at most 1%, at most 0.5% or at most 0.1% of the first liquid component by volume. In one example, the second liquid component at the second outlet comprises at most 10%, at most 5%, at most 3%, at most 2%, at most 1%, at most 0.5% or at most 0.1% of the first liquid component by volume. In one example, the first liquid component at the first outlet comprises at most 10%, at most 5%, at most 3%, at most 2%, at most 1%, at most 0.5% or at most 0.1% of the second liquid component by volume.

The respective conduits of the first set of N conduits are arranged to, at least in part, equalize flowrate ratios at the respective divisions. That is, the respective flowrates of the second liquid component through the respective conduits are similar. In this way, an efficiency of separation of the first liquid component and the second liquid component may be improved, for example at higher flowrates of the liquid via the inlet. In one example, the respective flowrates of the second liquid component through the respective conduits are within 75%, preferably within 50%, more preferably within 25% of the mean flowrate through the respective conduits. In one example, a difference between the maximum flowrate and the minimum flowrate through the respective conduits is at most 100%, preferably at most 75%, more preferably at most 50%, most preferably at most 25% of the minimum flowrate.

The respective conduits of the first set of N conduits are arranged to, at least in part, equalize flowrate ratios at the respective divisions by normalizing respective flowrate ratios (also known as split ratios) of the respective conduits of the first set of N conduits, wherein a flowrate ratio of a specific conduit of the first set of N conduits is the ratio of a flowrate of the liquid through the first passageway following the respective division to a flowrate of the second liquid component through the specific conduit. Thus, for a flowrate ratio of 1:1 (i.e. 50%), the flowrates in the first passageway and the specific conduit are equal while for a flowrate ratio of 10:1 (i.e. 9.1%), the flowrate in the first passageway is a factor of 10 greater than in the specific conduit. Separation of the first liquid component and the second liquid component may be improved by increasing the flowrate ratios. In one example, the respective flow rate ratios are in a range from 2:1 to 30:1, preferably in a range from 5:1 to 25:1, more preferably in a range from 8:1 to 20:1, most preferably in a range from 10:1 to 16:1. In one example, the respective flow rate ratios are in a range from 33% to 3%, preferably in a range from 17% to 3.5%, more preferably in a range from 11% to 4.5%, most preferably in a range from 9% to 6%. In one example, the respective flowrate ratios are within 50%, preferably within 25%, more preferably within 10% of the mean flowrate ratio. In one example, a difference between the maximum flowrate ratio and the minimum flowrate ratio is at most 30%, preferably at most 20%, more preferably at most 15%, most preferably at most 10% of the minimum flowrate ratio.

In one example, the respective conduits of the first set of N conduits are arranged to, at least in part, equalize flowrate ratios at the respective divisions by having respective lengths, cross-sectional areas, cross-sectional shapes and/or internal surfaces arranged to attenuate respective flowrates therethrough according to, at least in part, respective liquid pressures at the respective divisions.

In one example, a length of a conduit of the first set of N conduits is arranged to control a flowrate of the second liquid component therethrough. In one example, respective lengths of the respective conduits of the first set of N conduits are arranged to control respective flowrates of the second liquid component therethrough. In one example, the respective conduits of the first set of N conduits have respective lengths arranged to attenuate respective flowrates therethrough according to, at least in part, the respective liquid pressure at the respective divisions. In this way, respective flowrates of the second liquid component through the respective conduits may be normalized. In one example, a length of a conduit of the first set of N conduits is in a range from 0.1 to 100 mm, preferably in a range from 0.5 to 50 mm, more preferably in a range from 1 to 10 mm. In one example, an aspect ratio (i.e. a ratio of a length to a cross-section dimension such as width, height or diameter) of conduit of the first set of N conduits is in a range from 20 to 2000, preferably in a range from 50 to 1500, more preferably in a range from 100 to 1000.

In one example, a cross-sectional dimension, for example a width, a height, a diameter and/or a cross-sectional area, of a conduit of the first set of N conduits is relatively small, compared with the first passageway. In this way, an efficiency of separation of the second liquid component from the liquid may be increased. In one example, a cross-sectional dimension, for example a width, a height, a diameter and/or a cross-sectional area, is arranged to control a flowrate of the second liquid component therethrough. In one example, respective cross-sectional dimensions, for example a width, a height, a diameter and/or a cross-sectional area, of the respective conduits of the first set of N conduits are arranged to control respective flowrates of the second liquid component therethrough. In one example, the respective conduits of the first set of N conduits have respective cross-sectional dimensions, for example a width, a height, a diameter and/or a cross-sectional area, arranged to attenuate respective flowrates therethrough according to, at least in part, the respective liquid pressure at the respective divisions. In this way, respective flowrates of the second liquid component through the respective conduits may be normalized. In one example, a width, a height and/or a diameter of a conduit of the first set of N conduits is in a range from 1 μm to 50 μm, preferably in a range from 2 μm to 40 μm, more preferably in a range from 5 μm to 30 μm, for example 7.5 μm, 10 μm, 12.5 μm, 15 μm, 17.5 μm, 20 μm, 22.5 μm, 25 μm or 27.5 μm. In one example, a cross-sectional area of a conduit of the first set of N conduits is in a range from 1 μm² to 2500 μm², preferably in a range from 4 μm² to 1600 μm², more preferably in a range from 25 μm² to 900² μm, for example 10000 μm². In one example, each conduit of the first set of N conduits has similar, for example the same, cross-sectional dimensions. In this way, cost and/or complexity may be reduced. In one example, successive conduits of the first set of N conduits have successively larger cross-sectional dimensions. In this way, respective flowrates of the second liquid component therethrough may be normalized since smaller cross-sectional dimensions attenuate flows more than larger cross-sectional dimensions.

In one example, a cross-sectional shape of a conduit of the first set of N conduits is arranged to control a flowrate of the second liquid component therethrough. In this way, respective flowrates of the second liquid component through the respective conduits may be normalized. In one example, a cross-sectional shape of a conduit of the first set of N conduits is a symmetrical shape, for example a symmetric oval, a circle, a symmetric polygon such as a square or rectangle. In this way, flow characteristics of the liquid therein may be better controlled while a complexity and/or cost may be reduced. In one example, a cross-sectional shape of a conduit of the first set of N conduits is constant along at least a part of the length thereof, preferably along substantially the length thereof, for example along at least 50%, at least 60%, at least 70%, at least 80%, at least 90%, at least 95% or at least 97.5% of the length thereof. In this way, flow characteristics of the liquid therein may be better controlled while a complexity and/or cost may be reduced. In one example, internal corners of a conduit of the first set of N conduits are smooth and/or radiused. In this way, flow of the liquid therein may be more uniform, thereby reducing mixing of the liquid. In one example, a conduit of the first set of N conduits includes no internal corners. In one example, internal surfaces of a conduit of the first set of N conduits are smooth, having no protuberances or recesses. In this way, flow of the liquid therein may be more uniform, thereby reducing mixing of the liquid, and/or reducing unswept dead volumes.

In one example, a cross-sectional dimension, for example a width, a height, a diameter and/or a cross-sectional area, of a conduit of the first set of N conduits is constant along a length thereof. In one example, a conduit of the first set of N conduits tapers, for example uniformly, along a part a length thereof. In this way, stability of flow of the liquid therethrough may be improved. In one example, a conduit of the first set of N conduits tapers, for example uniformly, along a part a length thereof, wherein in a reduction in a cross-sectional dimension, for example a width, a height, a diameter and/or a cross-sectional area, is in a range from 10% to 90%, preferably in a range from 20% to 75%, more preferably in a range from 30% to 60%. In one example, a conduit of the first set of N conduits tapers, for example uniformly, along a part of the length thereof in a range from 5% to 90%, preferably in a range from 10% to 75%, more preferably in a range from 20% to 50% of the length. In one example, a conduit of the first set of N conduits tapers from and/or proximal from the inlet towards the first outlet. In one example, a cross-sectional dimension, for example a width, a height, a diameter and/or a cross-sectional area, of a conduit of the first set of N conduits along a remaining length (i.e. excluding the part of the length along which a conduit of the first set of N conduits tapers) is constant.

In one example, the respective conduits of the first set of N conduits divide from the first passageway at respective divisions from the inlet therealong on a same side, preferably only a same side, of the first passageway. In this way, the respective divisions for these respective conduits are mutually different. In one example, the respective conduits of the first set of N conduits divide from the first passageway at respective divisions from the inlet therealong on opposed sides of the first passageway, for example wherein the respective divisions are staggered (i.e. the respective divisions for these respective conduits are mutually different) and/or paired (i.e. the respective divisions for pairs of these respective conduits are the same).

Boustrophedonic Arrangement of Conduit

In one example, a conduit of the first set of N conduits is arranged, at least in part, boustrophedonically. That is, the conduit may be arranged in a meander, a zig-zag or a serpentine manner, alternately left to right then right to left, for example. In this way, an effective length of the conduit may be increased for a given size or net length of the conduit, thereby increasing a fluidic resistance thereof, as described above. Such a boustrophedonic arrangement of the conduit may provide relatively longer portions of the conduit arranged transversally to and alternately with relatively shorter portions of the conduit. The conduit may be arranged spirally or helically, so as to similarly increase a fluidic resistance thereof for a given size or net length of the microfluidic chamber. In one example, a conduit of the first set of N conduits is arranged, at least in part, boustrophedonically, having parallel legs of equal lengths. In one example, successive conduits of the first set of N conduits have successively fewer boustrophedonic parts thereof. In one example, the first conduit of the first set of N conduits is arranged, at least in part, boustrophedonically and the last conduit of the first set of N conduits is not arranged, at least in part, boustrophedonically.

Dead Volumes

In one example, the microfluidic device is arranged to reduce or avoid dead volumes, for example, by reducing or eliminating internal corners or recesses. Corners of the microfluidic device may be chamfered or radiused, to facilitate flow of the liquid and/or reduce or avoid dead volumes.

Flow of Biological Liquids

Flow of fluids, for example biological liquids such as blood, in microfluidic devices may deviate from theoretical flow models, due at least in part to particles, for example deformable particles, included in the fluids.

Flow of fluids, for example biological fluids such as blood, through a straight microchannel may result in deformable particles, for example leukocytes in blood, moving away from the microchannel walls due to an inertial lift effect if the Reynolds number is close to 1 and/or due to a viscous lift effect if the Reynolds number is lower. Both effects may arise due to the presence of the stationary microchannel walls and the interaction of a shear gradient on the deformable particles. A transitional regime may also exist in which both effects occur. A cell-free layer observed on the microchannel walls under certain conditions may depend also on particle-particle (i.e. inter-particle) interactions and/or a concentration of the particles.

Flow of fluids, for example biological fluids such as blood, through a non-straight microchannels, for example bifurcated microchannels or constricted microchannels, may be subject to other hydrodynamic effects including the Zweifach-Fung bifurcation effect and/or the constriction focusing effect, as described below.

Flow of fluids, for example biological fluids such as blood, through a bifurcated microchannel (i.e. a microchannel is branched or divided, equally or non-equally, into two microchannels), may result in the Zweifach-Fung bifurcation law or effect. The Zweifach-Fung bifurcation law is an empirical law relating to the behaviour of deformable particles at a bifurcation in a channel. At such a bifurcation, a cell in a flowing fluid tends to be transported into the branched channel having the higher flowrate, providing that a dimension of the cell is comparable to a dimension of the branched channel. Although this law was first proposed in the context of micro-circulation in the human body, it may also apply to in vitro flow, such as in microchannels. Further, this bifurcation law may be extended to apply to populations of cells flowing in microchannels tens of microns wide. However, generally the Zweifach-Fung law, as applied to microchannels, may be at most used to estimate lower limits for flowrate ratios at bifurcations, since the physics of flow, especially of biological liquids, in microchannels and limitations of the Zweifach-Fung law are not fully understood.

Flow of fluids, for example biological fluids such as blood, through a constricted microchannel (i.e. a microchannel including a constriction) may result in a focusing effect. For example, the first liquid component may be focused towards the microchannel walls and/or the second liquid component may be focused away from the microchannel walls, or vice versa, due, at least in part, to the constriction. This focusing effect may be relatively small in relatively wider microchannels, for example a 200 μm wide microchannel, and/or at relatively higher flowrates.

Constriction Members

In one example, the first passageway comprises a set of constriction members, wherein respective constriction members of the set of constriction members correspond with the respective conduits of the first set of N conduits. In other words, the first passageway includes one or more constrictions along its length. In this way, focusing of at least a part of the first liquid component and/or the second liquid component may be provided, as described above. For example, red blood cells may be focused after flowing through such a constriction, resulting in a substantially cell-free layer proximal the microchannel walls and a relatively cell-enriched stream centrally. In one example, the respective constriction members of the set of constriction members are arranged upstream of the respective conduits of the first set of N conduits. In other words, the constrictions in the first passageway are before the bifurcations such that focusing occurs before splitting of the flow. In this way, that liquid component focused relatively more centrally in the first passageway may tend to continue to flow therealong after the bifurcation while that liquid component focused relatively more proximal the walls of the first passageway may tend to flow into the bifurcated conduit. In this way, separation of the first liquid component and the second liquid component may be improved.

In one example, each constriction member has a length in a range from 0.1 mm to 10 mm, preferably in a range from 0.2 mm to 1 mm, more preferably in a range from 0.25 mm to 0.5 mm for example 0.3 mm. In one example, each constriction member has a width or diameter in a range from 10 μm to 100 μm, preferably in a range from 20 μm to 75 μm, more preferably in a range from 30 μm to 50 μm for example 38 μm. In one example, each constriction member has a height or diameter in a range from 10 μm to 100 μm, preferably in a range from 15 μm to 50 μm, more preferably in a range from 20 μm to 30 μm for example 25 μm.

In one example, each constriction member provides a constriction, relative to a cross-sectional area of the first passageway, in a range from 5% to 95%, preferably in a range from 10% to 90%, more preferably in a range from 25% to 75%, most preferably in a range from 35% to 65%, for example 50%. It should be understood that a constriction member providing a 50% constriction thus has a cross-sectional area of (100% −50%)=50% of the first passageway.

In one example, the set of constriction members are arranged proximal to and/or on a same side, preferably only a same side, of the first passageway. In this way, the first liquid component is urged towards this same side of the first passageway, thereby allowing improved separation of the second liquid component therefrom via the conduits. For example, during separation of blood, the cells are urged towards this same side of the first passageway, thereby enhancing a cell-free zone towards the opposed side of the first passageway during subsequent expansion in the set of expansion members, for example, thereby improving separation of the plasma from the cells via the conduits. At higher haematocrit levels, for example, when cell-cell interactions are higher, this is beneficial to improve separation efficiency.

In one example, the set of constriction members are arranged proximal to and/or on a same side, preferably only a same side, of the first passageway wherein the same side is of an inner radius of the first radius of curvature and/or the second radius of curvature, preferably wherein the first radius of curvature and the second radius of curvature are in a same direction, for example clockwise or counter-clockwise. By curving only in one direction, a second liquid component layer remains relatively stable while the centrifugal force tends to widen an inner layer of the second liquid component and reduce an outer layer thereof at the exit of the relatively large radii of curvature, which then corresponds with the effect due to the set of the set of constriction members are arranged proximal to and/or on a same side.

In one example, the set of constriction members are arranged proximal to and/or on a same side, preferably only a same side, of the first passageway and the respective conduits of the first set of N conduits divide from the first passageway at respective divisions from the inlet therealong on an opposed side, preferably only an opposed side, of the first passageway. In one example, the set of constriction members are arranged proximal to and/or on a same side, preferably only a same side, of the first passageway, wherein the same side is of an inner radius of the first radius of curvature and/or the second radius of curvature, preferably wherein the first radius of curvature and the second radius of curvature are in a same direction, and the respective conduits of the first set of N conduits divide from the first passageway at respective divisions from the inlet therealong on an opposed side, preferably only an opposed side, of the first passageway.

Expansion Members

In one example, the first passageway comprises a set of expansion members, wherein respective expansion members of the set of expansion members correspond with the respective conduits of the first set of N conduits and/or are arranged downstream thereof. In other words, the expansions in the first passageway may be after the bifurcations such that expansion occurs after splitting of the flow.

In one example, each expansion member has a length in a range from 0.1 mm to 10 mm, preferably in a range from 0.2 mm to 2 mm, more preferably in a range from 0.25 mm to 1 mm for example 0.5 mm. In one example, each expansion member has a width or diameter in a range from 30 μm to 300 μm, preferably in a range from 50 μm to 250 μm, more preferably in a range from 100 μm to 200 μm for example 150 μm. In one example, each expansion member has a height or diameter in a range from 10 μm to 100 μm, preferably in a range from 15 μm to 50 μm, more preferably in a range from 20 μm to 30 μm for example 25 μm.

In one example, each expansion member provides a expansion, relative to a cross-sectional area of the first passageway, in a range from 5% to 500%, preferably in a range from 25% to 300%, more preferably in a range from 35% to 200%, most preferably in a range from 45% to 65%, for example 50%. It should be understood that a expansion member providing a 50% expansion thus has a cross-sectional area of (100%+50%)=150% of the first passageway.

In one example, the set of constriction members are arranged proximal to and/or on a same side, preferably only a same side, of the first passageway and the set of set of expansion members downstream therefrom is arranged to expand towards an opposed side of the first passageway (i.e. away from the same side of the first passageway). In this way, the first liquid component is urged towards this same side of the first passageway, thereby allowing improved separation of the second liquid component therefrom via the conduits. For example, during separation of blood, the cells are urged towards this same side of the first passageway, thereby enhancing a cell-free zone towards the opposed side of the first passageway during subsequent expansion in the set of expansion members, for example, thereby improving separation of the plasma from the cells via the conduits. At higher haematocrit levels, for example, when cell-cell interactions are higher, this is beneficial to improve separation efficiency.

In one example, the set of constriction members are arranged proximal to and/or on a same side, preferably only a same side, of the first passageway, the set of set of expansion members downstream therefrom is arranged to expand towards an opposed side of the first passageway and the respective conduits of the first set of N conduits divide from the first passageway at respective divisions from the inlet therealong on the opposed side, preferably only the opposed side, of the first passageway.

In one example, the set of constriction members are arranged proximal to and/or on a same side, preferably only a same side, of the first passageway, wherein the same side is of an inner radius of the first radius of curvature and/or the second radius of curvature, preferably wherein the first radius of curvature and the second radius of curvature are in a same direction, the set of set of expansion members downstream therefrom is arranged to expand towards an opposed side of the first passageway and the respective conduits of the first set of N conduits divide from the first passageway at respective divisions from the inlet therealong on the opposed side, preferably only the opposed side, of the first passageway.

Acute Angle

In one example, the respective conduits divide from the first passageway at the respective divisions by being arranged at respective acute angles thereto, wherein respective intersections of the respective conduits and the first passageway at the respective divisions define arcuate flow paths of the second liquid component. In this way, a cell-free zone may be enhanced, as described below in more detail. In one example, the respective acute angles are in a range from 1° to 89°, preferably in a range from 15° to 75°, more preferably in a range from 30° to 60°, for example 45°. By reducing the acute angle, the enhancement of the cell-free zone may be further increased.

In one example, the first passageway comprises a set of first passageways and wherein respective first passageways of the set of first passageways divide from the inlet and the first outlet.

In one example, the first outlet is fluidically coupled to the inlet via a second passageway; and the second outlet is fluidically coupled to the second passageway via a second set of M conduits, wherein M is a positive integer greater than 1, wherein respective conduits of the second set of M conduits divide from the second passageway at respective divisions from the inlet therealong;

wherein the respective conduits of the second set of M conduits are arranged to, at least in part, equalize flowrate ratios at the respective divisions.

The second passageway and/or the second set of M conduits may be as described with respect to the first passageway and/or the first set of N conduits, respectively. In one example, N and M are equal.

In one example, the microfluidic device comprises a first outlet passageway fluidically coupled to the second outlet and to the first set of N conduits, wherein the second outlet is fluidically coupled to the first passageway via the first set of N conduits and the first outlet passageway.

In one example, the microfluidic device comprises a second outlet passageway fluidically coupled to the second outlet and to the second set of M conduits, wherein the second outlet is fluidically coupled to the second passageway via the second set of M conduits and the second outlet passageway.

In one example, the inlet, the first outlet and the second outlet are arranged collinearly, thereby defining an axis.

In one example, the first passageway and/or the first set of N conduits is arranged symmetrically about the axis. In one example, the second passageway and/or the second set of M conduits is a reflection of the first passageway and/or the first set of N conduits, in which the axis is a mirror line, thereby defining a heart-shape (i.e. a cardioid).

In one example, the microfluidic device is a blood separation device and the second liquid component comprises separated plasma.

The microfluidic device may be chemically and/or biologically inert. That is, the microfluidic device may be compatible with biological samples, for example. Alternatively, the microfluidic device may be chemically and/or biologically active and/or reactive. For example, the microfluidic device may react with biological samples. For example, the microfluidic device may comprise a catalyst. The microfluidic device may comprise a material having such properties. A wall of the microfluidic device may comprise such a material. An internal surface of the microfluidic device may comprise such a material. For example, the microfluidic device may comprise a polymeric composition comprising a polymer, a metal such as an alloy and/or a ceramic. For example, the microfluidic device may a polymeric composition comprising a polymer such as poly (methyl methacrylate) (PMMA). For example, the microfluidic device may comprise a metal such as a stainless steel such as 316 stainless steel. For example, the microfluidic device may comprise a ceramic such as silicon dioxide. An internal surface of the microfluidic device may comprise a coating of such a material. Generally, such a material is relatively incompressible, in use, improving fluid flow, for example reliability thereof, in the microfluidic device.

The second aspect of the invention provides an apparatus arranged to control a microfluidic device according to the first aspect. The apparatus may comprise a controller, one or more pumps or injectors, one or more valves, one or more heaters and/or one or more detectors. The controller may be arranged to control at least one of the one or more pumps, at least one of the one or more valves and/or at least one of the one or more detectors. The controller may be arranged to control a flow rate of the liquid into and/or through the microfluidic device. For example, the controller may be arranged to control one of the one or more pumps or injectors to pump or inject the liquid into the microfluidic device at a flow rate in a range from 1 to 100 ml/hr, preferably in a range from 2 to 50 ml/hr, more preferably in a range from 5 to 30 ml/hr.

The third aspect provides a microfluidic system comprising an apparatus according to the second aspect and a microfluidic device according to the first aspect.

The fourth aspect provides a method of operating a microfluidic system according to the third aspect.

The fifth aspect provides a lab-on-a-chip device comprising a microfluidic device according to the first aspect.

### BRIEF DESCRIPTION OF THE DRAWINGS

For a better understanding of the invention, and to show how exemplary embodiments of the same may be brought into effect, reference will be made, by way of example only, to the accompanying diagrammatic Figures, in which:

FIG. 1 schematically depicts a microfluidic device according to an exemplary embodiment;

FIG. 2 schematically depicts a microfluidic device according to an exemplary embodiment;

FIG. 3 schematically depicts the microfluidic device of FIG. 2, in more detail;

FIG. 4 schematically depicts the microfluidic device of FIG. 2, in more detail;

FIG. 5 schematically depicts a microfluidic device according to an exemplary embodiment;

FIG. 6 schematically depicts the microfluidic device of FIG. 5, in more detail;

FIG. 7 schematically depicts the microfluidic device of FIG. 5, in more detail;

FIG. 8 schematically depicts a microfluidic device according to an exemplary embodiment;

FIG. 9 schematically depicts the microfluidic device of FIG. 8, in more detail;

FIG. 10 schematically depicts the microfluidic device of FIG. 8, in more detail;

FIG. 11 schematically depicts the microfluidic device of FIG. 5, in more detail, in use;

FIG. 12 is a graph showing results for microfluidic devices according to exemplary embodiments compared with a conventional microfluidic device;

FIG. 13A is a graph showing results for a conventional microfluidic device and FIG. 13B is a graph showing results for a microfluidic device according to exemplary embodiment; and

FIG. 14 is a graph showing results for microfluidic devices according to exemplary embodiments compared with conventional microfluidic devices.

### DETAILED DESCRIPTION OF THE DRAWINGS

Generally, like reference signs indicate like features.

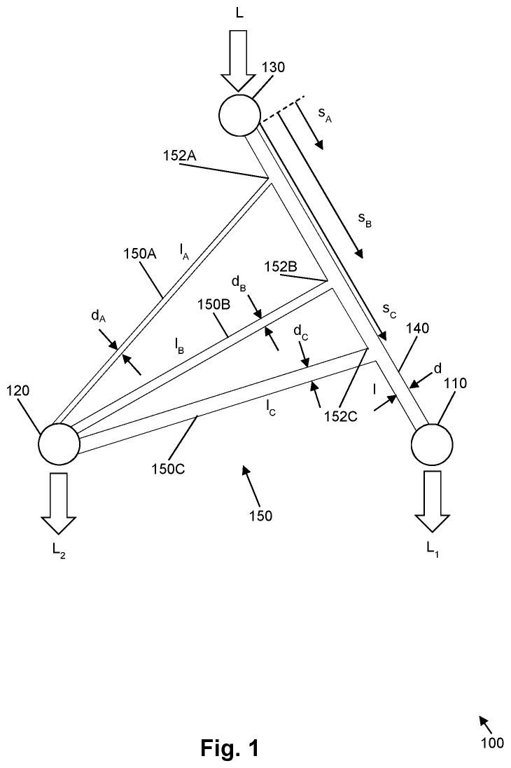

FIG. 1 schematically depicts a microfluidic device 100 according to an exemplary embodiment. Particularly, FIG. 1 shows a plan view of the microfluidic device 100.

Particularly, the microfluidic device 100 is for separating a liquid L into first and second liquid components L₁, L₂ thereof. The microfluidic device 100 comprises an inlet 130 for receiving the liquid therethrough. The microfluidic device 100 comprises a first outlet 110 for the first liquid component L₁, wherein the first outlet 110 is fluidically coupled to the inlet 130 via a first passageway 140. The microfluidic device 100 comprises a second outlet 120 for the second liquid component L₂, wherein the second outlet 120 is fluidically coupled to the first passageway 140 via a first set of N conduits 150 (150A, 150B, 150C), wherein N is a positive integer greater than 1, wherein respective conduits 150A, 150B, 150C of the first set of N conduits 150 divide from the first passageway 140 at respective divisions 152 (152A, 152B, 152C) from the inlet 130 therealong. The respective conduits 150A, 150B, 150C of the first set of N conduits 150 are arranged to, at least in part, equalize flowrate ratios at the respective divisions 152 (152A, 152B, 152C).

The first set of N conduits 150 consists of 3 conduits (i.e. N=3). The first conduit 150A is divided from the first passageway 140 at a spacing s_{A} from the inlet 130 therealong, thereby providing the first division 152A, specifically a first bifurcation 152A. Similarly, the second conduit 150B is divided from the first passageway 140 at a spacing s_{B} from the inlet 130 therealong, thereby providing the second division 152B, specifically a second bifurcation 152B. Similarly, the third conduit 150C is divided from the first passageway 140 at a spacing s_{C} from the inlet 130 therealong, thereby providing the third division 152C, specifically a third bifurcation 152C.

The first passageway 140 is straight, having a length I, and has a constant circular cross-sectional area, having a diameter d. The first conduit 150A is straight, having a length l_{A}, and has a constant circular cross-sectional area, having a diameter d_{A}. The second conduit 150B is straight, having a length l_{B}, and has a constant circular cross-sectional area, having a diameter d_{B}. The third conduit 150C is straight, having a length l_{C}, and has a constant circular cross-sectional area, having a diameter d_{C}. The diameter d>>the diameter d_{C}>the diameter d_{B} >the diameter d_{A}. The length l_{A}˜the length l_{C}>the length l_{B}.

Particularly, the respective conduits 150A, 150B, 150C of the first set of N conduits 150 are arranged to, at least in part, equalize flowrate ratios at the respective divisions 152 (152A, 152B, 152C) by having respective lengths l_{A}, l_{B}, l_{C} and cross-sectional diameters d_{A}, d_{B}, d_{C} (i.e. cross-sectional areas since circular) arranged to attenuate respective flowrates therethrough according to, at least in part, respective liquid pressures at the respective divisions 152 (152A, 152B, 152C), as described above.

The inlet 130, the first outlet 110 and the second outlet 120 are mutually equispaced, arranged in an equilateral triangle.

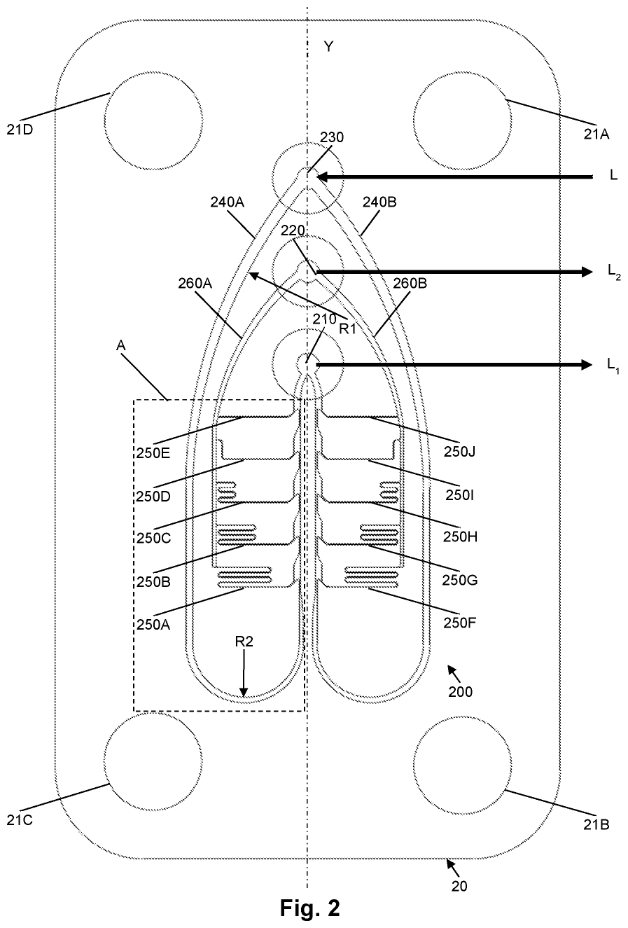

FIG. 2 schematically depicts a microfluidic device 200 according to an exemplary embodiment. Particularly, FIG. 2 shows a plan view of the microfluidic device 200.

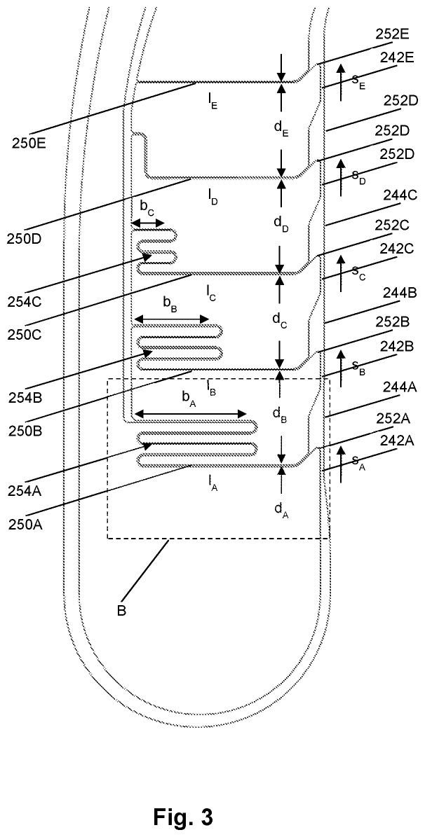

FIG. 3 schematically depicts the microfluidic device 200 of FIG. 2, in more detail. Particularly, FIG. 3 shows an enlarged portion of region A of FIG. 2.

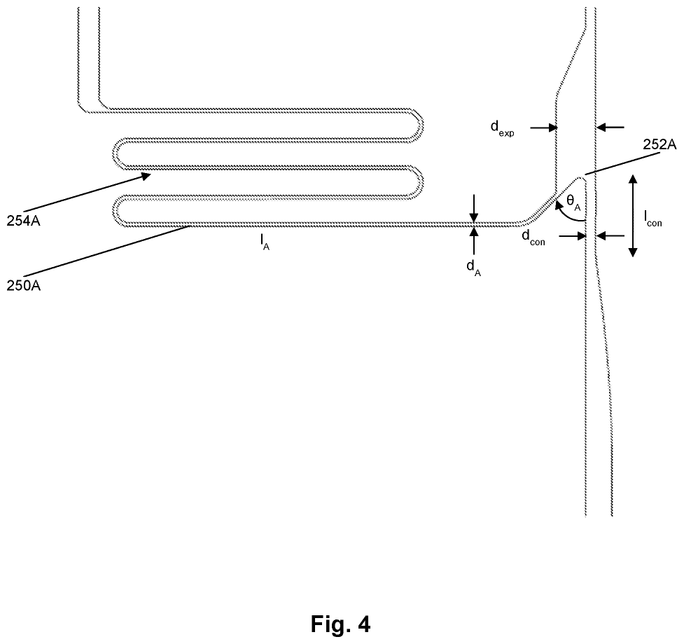

FIG. 4 schematically depicts the microfluidic device 200 of FIG. 2, in more detail. Particularly, FIG. 4 shows an enlarged portion of region B of FIG. 3.

Particularly, the microfluidic device 200 is for separating a liquid L into first and second liquid components L₁, L₂ thereof. The microfluidic device 200 comprises an inlet 230 for receiving the liquid therethrough. The microfluidic device 200 comprises a first outlet 210 for the first liquid component L₁, wherein the first outlet 210 is fluidically coupled to the inlet 230 via a first passageway 240A. The microfluidic device 200 comprises a second outlet 220 for the second liquid component L₂, wherein the second outlet 220 is fluidically coupled to the first passageway 240A via a first set of N conduits 250 (250A, 250B, 250C, 250D, 250E), wherein N is a positive integer greater than 1, wherein respective conduits 250A, 250B, 250C, 250D, 250E of the first set of N conduits 250 divide from the first passageway 240A at respective divisions 252 (252A, 252B, 252C, 252D, 252E) from the inlet 230 therealong. The respective conduits 250A, 250B, 250C, 250D, 250E of the first set of N conduits 250 are arranged to, at least in part, equalize flowrate ratios at the respective divisions 252 (252A, 252B, 252C, 252D, 252E).

The microfluidic device 200 is for separating whole blood. Preferably, the microfluidic device 200 is for separating diluted whole blood, for example whole blood diluted by a ratio of at most 10:1, preferably at most 5:1 more preferably at most 2:1, most preferably at most 1:1.

The first set of N conduits 250 consists of 5 conduits (i.e. N=5). The first conduit 250A is divided from the first passageway 240A at a spacing s_{A} from the inlet 230 therealong, thereby providing the first division 252A, specifically a bifurcation 252A. Similarly, the second conduit 250B is divided the first passageway 240A at a spacing s_{B} from the inlet 230 therealong, thereby providing the second division 252B, specifically a bifurcation 252B. Similarly, the third conduit 250C is divided from the first passageway 240A at a spacing s_{C} from the inlet 230 therealong, thereby providing the third division 252C, specifically a bifurcation 252C. Similarly, the fourth conduit 250D is divided from the first passageway 240A at a spacing s_{D} from the inlet 230 therealong, thereby providing the fourth division 252D, specifically a bifurcation 252D. Similarly, the fifth conduit 250E is divided from the first passageway 240A at a spacing s_{E} from the inlet 230 therealong, thereby providing the fifth division 252E, specifically a bifurcation 252E.

In this example, the respective conduits 250A, 250B, 250C, 250D, 250E of the first set of N conduits 250 have respective lengths l_{A}, l_{B}, l_{C}, l_{D}, l_{E} arranged to attenuate respective flowrates therethrough according to, at least in part, the respective liquid L pressure at the respective divisions 252 (252A, 252B, 252C, 252D, 252E). In this example, the length l_{A}>the length l_{B} >the length l_{C}>the length l_{D}>the length l_{E}. In this example, the length l_{A} is 6.75 mm, the length l_{B} is 5.4 mm the length l_{C} is 3.57 mm, the length l_{D} is 2.16 mm and the length l_{E} is 1.71 mm. In this example, the respective conduits 250A, 250B, 250C, 250D, 250E have the same constant rectangular cross-sectional areas, having respective equal widths d_{A}, d_{B}, d_{C}, d_{D}, d_{E} of 15 μm and respective equal heights of 20 μm.

In this example, the first passageway 240A curves from the inlet towards the first outlet along a first part of a length thereof, wherein a first radius of curvature R1 of the first part is 10 mm. In this example, the first passageway 240A tapers along the first part of the length, from a width of 270 μm to a width of 100 μm over the first part of the length, wherein the first part of the length has a length of 15 mm. In this example, the first passageway 240A curves from the inlet towards the first outlet along a second part of a length thereof, wherein a second radius of curvature R2 of the second part is 1.3 mm. In this example, the first passageway 240A has a constant cross-sectional area along the second part of the length, having a constant width of 100 μm. In this example, the first passageway 240A is linear along a third part of a length thereof, between the first part of the length and the second part of the length, wherein the first passageway 240A has a constant cross-sectional area along the third part of the length, having a constant width of 100 μm. In this example, the first passageway 240A is arranged to surround, at least in part, the first set of N conduits 250.

In this example, the first passageway 240A comprises a set of constriction members 242 (242A, 242B, 242C, 242D, 242E), wherein respective constriction members 242A, 242B, 242C, 242D, 242E of the set of constriction members 242 correspond with the respective conduits 250A, 250B, 250C, 250D, 250E of the first set of N conduits 250. The respective constriction members 242A, 242B, 242C, 242D, 242E are arranged upstream (i.e. with respect to flow of the liquid L) of the respective conduits 250A, 250B, 250C, 250D, 250E. The respective constriction members 242A, 242B, 242C, 242D, 242E are arranged proximal to, and upstream of, the respective divisions 252A, 252B, 252C, 252D, 252E. In this example, the respective constriction members 242A, 242B, 242C, 242D, 242E of the set of constriction members 242 are similar. In this example, the respective constriction members 242A, 242B, 242C, 242D, 242E of the set of constriction members 242 have the same constant rectangular cross-sectional areas, having respective equal widths d_{con} of 38 μm, heights of 20 μm and lengths of 0.302 mm.

In this example, the first passageway 240A comprises a set of expansion members 244 (244A, 244B, 244C, 244D), wherein respective expansion members 244A, 244B, 244C, 244D of the set of expansion members 240A correspond with the respective conduits 250A, 250B, 250C, 250D of the first set of N conduits 250. The respective expansion members 244A, 244B, 244C, 244D are arranged downstream (i.e. with respect to flow of the liquid L) of the respective conduits 250A, 250B, 250C, 250D. The respective expansion members 244A, 244B, 244C, 244D are arranged proximal to, and downstream of, the respective divisions 252A, 252B, 252C, 252D. In this example, the respective expansion members 244A, 244B, 244C, 244D of the set of expansion members 240A are similar. In this example, the respective expansion members 244A, 244B, 244C, 244D of the set of expansion members 240A have the same rectangular cross-sectional areas, having respective equal widths dₑₓₚ of 153 μm and heights of 20 μm. An expansion member is not provided corresponding with the last conduit 250E of the first set of N conduits 250.

In this example, the conduit 250A of the first set of N conduits 250 is arranged at an acute angle θ_{A} to the first passageway 240. In this example, the acute angle θ_{A} is approximately 45°. The respective conduits 250B, 250C, 250D, 250E of the first set of N conduits 250 are arranged similarly at respective acute angles θ_{B}, θ_{C}, θ_{D}, θ_{E} to the first passageway 240, in which the angle θ_{A}=θ_{B}=θ_{C}=θ_{D}=θ_{E}.

In this example, the first passageway 240A defines a linear flow path of the liquid via the respective divisions, wherein a wall, for example opposed to the first set of N conduits 250, of the first passageway 240A extending between the respective divisions 252 is linear.

In this example, the conduit 250A of the first set of N conduits 250 is arranged boustrophedonically, having three parallel legs 254A of equal lengths b_{A}. In this example, the conduit 250B of the first set of N conduits 250 is arranged boustrophedonically, having three parallel legs 254B of equal lengths b_{B}. In this example, the conduit 250C of the first set of N conduits 250 is arranged boustrophedonically, having three parallel legs 254C of equal lengths b_{C}. In this example, the length b_{A}>the length b_{B}>the length b_{C}. In this example, the conduits 250D, 250E of the first set of N conduits 250 are not arranged boustrophedonically.

In this example, the inlet 230, the first outlet 210 and the second outlet 220 are arranged collinearly, thereby defining an axis Y.

In this example, the first outlet 210 is fluidically coupled to the inlet 230 via a second passageway 240B. In this example, the second 220 outlet is fluidically coupled to the second passageway 240B via a second set of M conduits 250 (250F, 250G, 250H, 250I, 250J), wherein M is a positive integer greater than 1, wherein respective conduits of the second set of M conduits 250 (250F, 250G, 250H, 250I, 250J) divide from the second passageway 240B at respective divisions 252 (252F, 252G, 252H, 252I, 252J) from the inlet 230 therealong. In this example, the respective conduits of the second set of M conduits 250 (250F, 250G, 250H, 250I, 250J) are arranged to, at least in part, equalize flowrate ratios at the respective divisions 252 (252F, 252G, 252H, 252I, 252J).

The second passageway 240B and the second set of M conduits 250 (250F, 250G, 250H, 250I, 250J) are as described with respect to the first passageway 240A and the first set of N conduits 250 (250A, 250B, 250C, 250D, 250E), respectively. In this example, N and M are equal to five.

In this example, the microfluidic device 200 comprises a first outlet passageway 260A fluidically coupled to the second outlet 220 and to the first set of N conduits 250 (250A, 250B, 250C, 250D, 250E), wherein the second outlet 220 is fluidically coupled to the first passageway 240A via the first set of N conduits 250 (250A, 250B, 250C, 250D, 250E) and the first outlet passageway 260A.

In this example, the microfluidic device 200 comprises a second outlet passageway 260B fluidically coupled to the second outlet 220 and to the second set of M conduits 250 (250F, 250G, 250H, 250I, 250J), wherein the second outlet 220 is fluidically coupled to the second passageway 240B via the second set of M conduits 250 (250F, 250G, 250H, 250I, 250J) and the second outlet passageway 260B.

In this example, the first passageway 240A and the first set of N conduits 250 (250A, 250B, 250C, 250D, 250E) are arranged symmetrically about the axis Y with respect to the second passageway 240B and the second set of M conduits 250 (250F, 250G, 250H, 250I, 250J). The second passageway 240B is a reflection of the first passageway 240A, in which the axis Y is a mirror line, thereby defining a heart-shape. The respective conduits 250F, 250G, 250H, 250I, 250J are respective reflections of the respective conduits 250A, 250B, 250C, 250D, 250E, in which the axis Y is the mirror line.

In this example, the microfluidic device 200 is provided on a rectangular lab-on-a-chip device 20, having four (4) apertures 21A, 21B, 21C, 21D therethrough provided at corners thereof for securing in use. Alternative methods of securing in use are known.

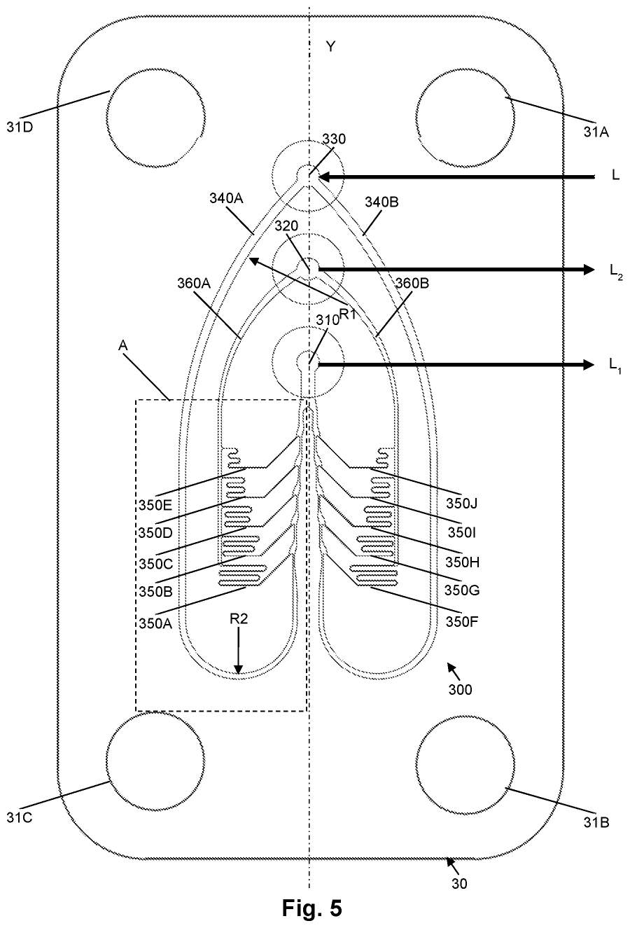

FIG. 5 schematically depicts a microfluidic device 300 according to an exemplary embodiment. Particularly, FIG. 5 shows a plan view of the microfluidic device 300.

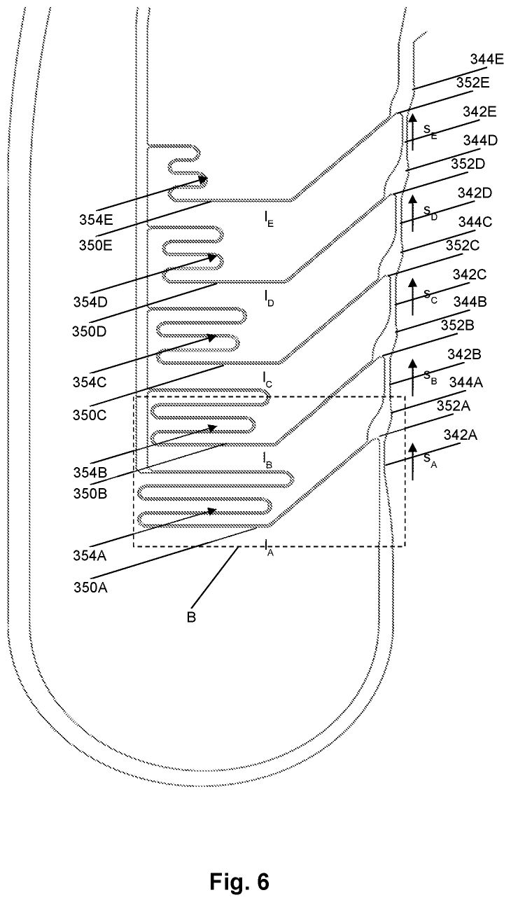

FIG. 6 schematically depicts the microfluidic device 300 of FIG. 5, in more detail. Particularly, FIG. 3 shows an enlarged portion of region A of FIG. 5.

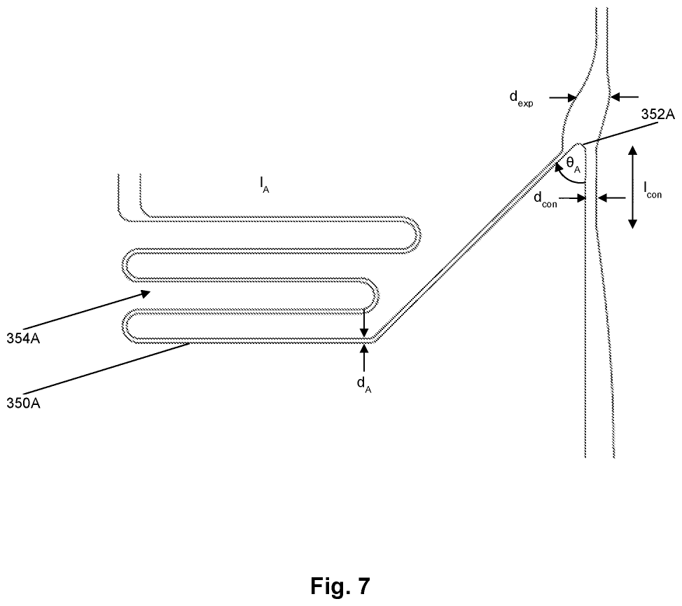

FIG. 7 schematically depicts the microfluidic device 300 of FIG. 5, in more detail. Particularly, FIG. 7 shows an enlarged portion of region B of FIG. 6.

Particularly, the microfluidic device 300 is for separating a liquid L into first and second liquid components L₁, L₂ thereof. The microfluidic device 300 comprises an inlet 330 for receiving the liquid therethrough. The microfluidic device 300 comprises a first outlet 310 for the first liquid component L₁, wherein the first outlet 310 is fluidically coupled to the inlet 330 via a first passageway 340A. The microfluidic device 300 comprises a second outlet 320 for the second liquid component L₂, wherein the second outlet 320 is fluidically coupled to the first passageway 340A via a first set of N conduits 350 (350A, 350B, 350C, 350D, 350E), wherein N is a positive integer greater than 1, wherein respective conduits 350A, 350B, 350C, 350D, 350E of the first set of N conduits 350 divide from the first passageway 340A at respective divisions 352 (352A, 352B, 352C, 352D, 352E) from the inlet 330 therealong. The respective conduits 350A, 350B, 350C, 350D, 350E of the first set of N conduits 350 are arranged to, at least in part, equalize flowrate ratios at the respective divisions 352 (352A, 352B, 352C, 352D, 352E).

Generally, the microfluidic device 300 is as described with respect to the microfluidic device 200.

The microfluidic device 300 is for separating whole blood. Preferably, the microfluidic device 300 is for separating diluted whole blood, for example whole blood diluted by a ratio of at most 10:1, preferably at most 5:1 more preferably at most 3:1, most preferably at most 1:1.

The first set of N conduits 350 consists of 5 conduits (i.e. N=5). The first conduit 350A is divided from the first passageway 340A at a spacing s_{A} from the inlet 330 therealong, thereby providing the first division 352A, specifically a bifurcation 352A. Similarly, the second conduit 350B is divided the first passageway 340A at a spacing s_{B} from the inlet 330 therealong, thereby providing the second division 352B, specifically a bifurcation 352B. Similarly, the third conduit 350C is divided from the first passageway 340A at a spacing s_{C} from the inlet 330 therealong, thereby providing the third division 352C, specifically a bifurcation 352C. Similarly, the fourth conduit 350D is divided from the first passageway 340A at a spacing s_{D} from the inlet 330 therealong, thereby providing the fourth division 352D, specifically a bifurcation 352D. Similarly, the fifth conduit 350E is divided from the first passageway 340A at a spacing s_{E} from the inlet 330 therealong, thereby providing the fifth division 352E, specifically a bifurcation 352E.

In this example, the respective conduits 350A, 350B, 350C, 350D, 350E of the first set of N conduits 350 have respective lengths l_{A}, l_{B}, l_{C}, l_{D}, l_{E} arranged to attenuate respective flowrates therethrough according to, at least in part, the respective liquid L pressure at the respective divisions 352 (352A, 352B, 352C, 352D, 352E). In this example, the length l_{A}>the length l_{B}>the length l_{C}>the length l_{D}>the length l_{E}. In this example, the length l_{A} is 6.75 mm, the length l_{B} is 5.4 mm the length l_{C} is 3.57 mm, the length l_{D} is 2.16 mm and the length l_{E} is 1.71 mm. In this example, the respective conduits 350A, 350B, 350C, 350D, 350E have the same constant rectangular cross-sectional areas, having respective equal widths d_{A}, d_{B}, d_{C}, d_{D}, d_{E} of 15 μm and respective equal heights of 20 μm.

In this example, the first passageway 340A curves from the inlet towards the first outlet along a first part of a length thereof, wherein a first radius of curvature R1 of the first part is 10 mm. In this example, the first passageway 340A tapers along the first part of the length, from a width of 370 μm to a width of 100 μm over the first part of the length, wherein the first part of the length has a length of 15 mm. In this example, the first passageway 340A curves from the inlet towards the first outlet along a second part of a length thereof, wherein a second radius of curvature R2 of the second part is 1.3 mm. In this example, the first passageway 340A has a constant cross-sectional area along the second part of the length, having a constant width of 100 μm. In this example, the first passageway 340A is linear along a third part of a length thereof, between the first part of the length and the second part of the length, wherein the first passageway 340A has a constant cross-sectional area along the third part of the length, having a constant width of 100 μm. In this example, the first passageway 340A is arranged to surround, at least in part, the first set of N conduits 350.

In this example, the first passageway 340A comprises a set of constriction members 342 (342A, 342B, 342C, 342D, 342E), wherein respective constriction members 342A, 342B, 342C, 342D, 342E of the set of constriction members 342 correspond with the respective conduits 350A, 350B, 350C, 350D, 350E of the first set of N conduits 350. The respective constriction members 342A, 342B, 342C, 342D, 342E are arranged upstream (i.e. with respect to flow of the liquid L) of the respective conduits 350A, 350B, 350C, 350D, 350E. The respective constriction members 342A, 342B, 342C, 342D, 342E are arranged proximal to, and upstream of, the respective divisions 352A, 352B, 352C, 352D, 352E. In this example, the respective constriction members 342A, 342B, 342C, 342D, 342E of the set of constriction members 342 are similar. In this example, the respective constriction members 342A, 342B, 342C, 342D, 342E of the set of constriction members 342 have the same constant rectangular cross-sectional areas, having respective equal widths d_{con} of 38 μm, heights of 20 μm and lengths of 0.302 mm.

In this example, the first passageway 340A comprises a set of expansion members 344 (344A, 344B, 344C, 344D, 344E), wherein respective expansion members 344A, 344B, 344C, 344D, 344E of the set of expansion members 340A correspond with the respective conduits 350A, 350B, 350C, 350D, 350E of the first set of N conduits 350. The respective expansion members 344A, 344B, 344C, 344D, 344E are arranged downstream (i.e. with respect to flow of the liquid L) of the respective conduits 350A, 350B, 350C, 350D, 350E. The respective expansion members 344A, 344B, 344C, 344D, 344E are arranged proximal to, and downstream of, the respective divisions 352A, 352B, 352C, 352D, 352E. In this example, the respective expansion members 344A, 344B, 344C, 344D, 344E of the set of expansion members 340A are similar. In this example, the respective expansion members 344A, 344B, 344C, 344D, 344E of the set of expansion members 340A have the same non-constant rectangular cross-sectional areas, having widths that enlarge smoothly and arcuately away from the respective divisions 352A, 352B, 352C, 352D, 352E to respective maximum widths dₑₓₚ and reduce smoothly and arcuately thereafter towards the respective constriction members 342A, 342B, 342C, 342D, 342E.

In this example, the conduit 350A of the first set of N conduits 350 is arranged at an acute angle θ_{A} to the first passageway 340. In this example, the acute angle θ_{A} is approximately 45°. The respective conduit 350B, 350C, 350D, 350E of the first set of N conduits 350 are arranged similarly at respective acute angles θ_{B}, θ_{C}, θ_{D}, θ_{E} to the first passageway 340, in which the angle θ_{A}=θ_{B}=θ_{C}=θ_{D}=θ_{E}.

In this example, the first passageway 340A defines a non-linear flow path of the liquid via the respective divisions, wherein a wall, for example opposed to the first set of N conduits 350, of the first passageway 340A extending between the respective divisions 352 is non-linear. Particularly, the wall, opposed to the first set of N conduits 350, of the first passageway 340A extending between the respective divisions 352 comprises alternating linear parts through the constriction members 342 (342A, 342B, 342C, 342D, 342E) and smoothly curved parts through the expansion members 344 (344A, 344B, 344C, 344D, 344E) therebetween, in which the smoothly curved parts are similar.

In this example, the conduit 350A of the first set of N conduits 350 is arranged boustrophedonically, having three parallel legs 354A of equal lengths b_{A}. In this example, the conduit 350B of the first set of N conduits 350 is arranged boustrophedonically, having three parallel legs 354B of equal lengths b_{B}. In this example, the conduit 350C of the first set of N conduits 350 is arranged boustrophedonically, having three parallel legs 354C of equal lengths b_{C}. In this example, the conduit 350D of the first set of N conduits 350 is arranged boustrophedonically, having three parallel legs 354D of equal lengths b_{D}. In this example, the conduit 350E of the first set of N conduits 350 is arranged boustrophedonically, having three parallel legs 354E of equal lengths b_{E}. In this example, the length b_{A}>the length b_{B}>the length b_{C}>the length b_{D}>the length b_{E}.

In this example, the inlet 330, the first outlet 310 and the second outlet 320 are arranged collinearly, thereby defining an axis Y.

In this example, the first outlet 310 is fluidically coupled to the inlet 330 via a second passageway 340B. In this example, the second 320 outlet is fluidically coupled to the second passageway 340B via a second set of M conduits 350 (350F, 350G, 350H, 350I, 350J), wherein M is a positive integer greater than 1, wherein respective conduits of the second set of M conduits 350 (350F, 350G, 350H, 350I, 350J) divide from the second passageway 340B at respective divisions 352 (352F, 352G, 352H, 352I, 352J) from the inlet 330 therealong. In this example, the respective conduits of the second set of M conduits 350 (350F, 350G, 350H, 350I, 350J) are arranged to, at least in part, equalize flowrate ratios at the respective divisions 352 (352F, 352G, 352H, 352I, 352J).

The second passageway 340B and the second set of M conduits 350 (350F, 350G, 350H, 350I, 350J) are as described with respect to the first passageway 340A and the first set of N conduits 350 (350A, 350B, 350C, 350D, 350E), respectively. In this example, N and M are equal to five.

In this example, the microfluidic device 300 comprises a first outlet passageway 360A fluidically coupled to the second outlet 320 and to the first set of N conduits 350 (350A, 350B, 350C, 350D, 350E), wherein the second outlet 320 is fluidically coupled to the first passageway 340A via the first set of N conduits 350 (350A, 350B, 350C, 350D, 350E) and the first outlet passageway 360A.

In this example, the microfluidic device 300 comprises a second outlet passageway 360B fluidically coupled to the second outlet 320 and to the second set of M conduits 350 (350F, 350G, 350H, 350I, 350J), wherein the second outlet 320 is fluidically coupled to the second passageway 340B via the second set of M conduits 350 (350F, 350G, 350H, 350I, 350J) and the second outlet passageway 360B.

In this example, the first passageway 340A and the first set of N conduits 350 (350A, 350B, 350C, 350D, 350E) are arranged symmetrically about the axis Y with respect to the second passageway 340B and the second set of M conduits 350 (350F, 350G, 350H, 350I, 350J). The second passageway 340B is a reflection of the first passageway 340A, in which the axis Y is a mirror line, thereby defining a heart-shape. The respective conduits 350F, 350G, 350H, 350I, 350J are respective reflections of the respective conduits 350A, 350B, 350C, 350D, 350E, in which the axis Y is the mirror line.

In this example, the microfluidic device 300 is provided on a rectangular lab-on-a-chip device 30, having four (4) apertures 31A, 31B, 31C, 31D therethrough provided at corners thereof for securing in use. Alternative methods of securing in use are known.

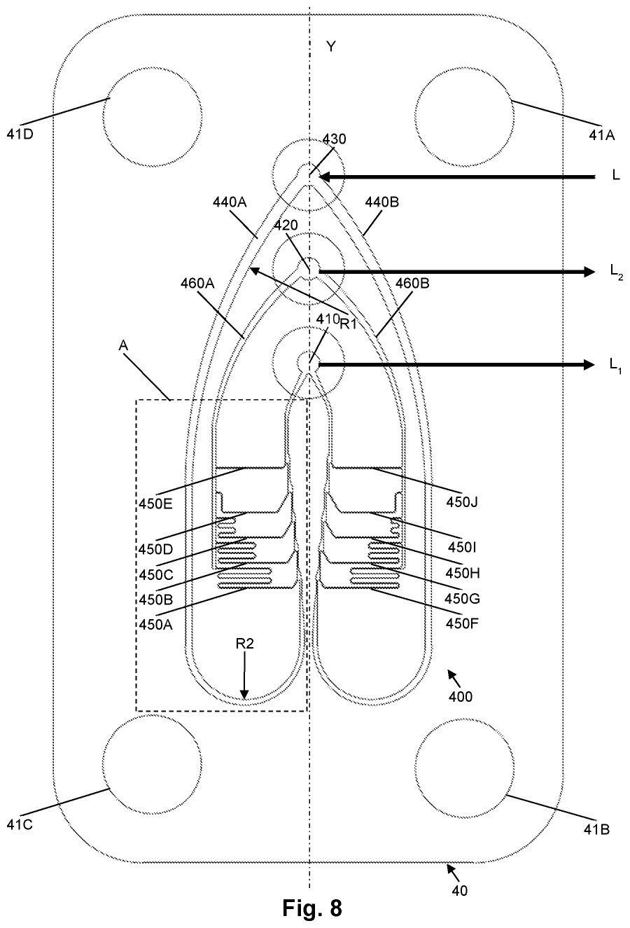

FIG. 8 schematically depicts a microfluidic device 400 according to an exemplary embodiment. Particularly, FIG. 8 shows a plan view of the microfluidic device 400.

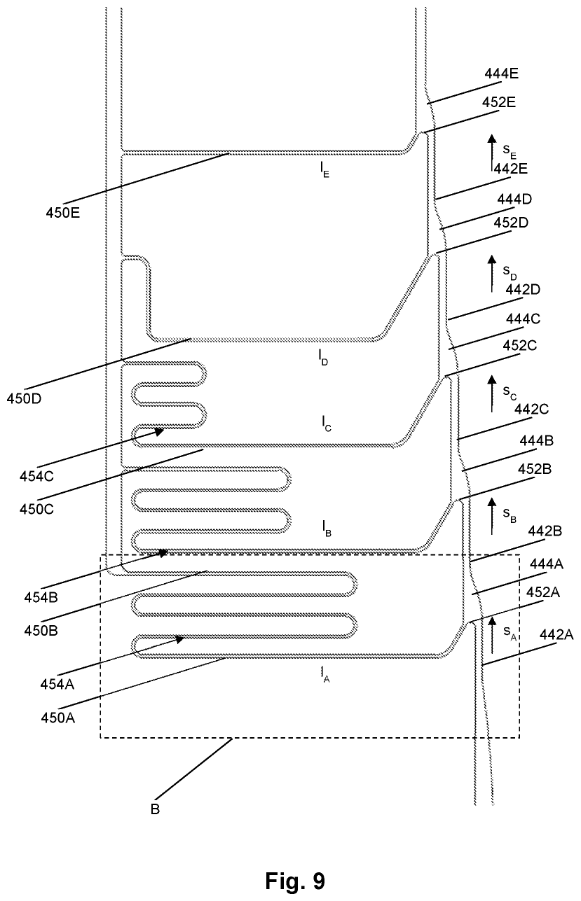

FIG. 9 schematically depicts the microfluidic device 400 of FIG. 8, in more detail. Particularly, FIG. 9 shows an enlarged portion of region A of FIG. 8.

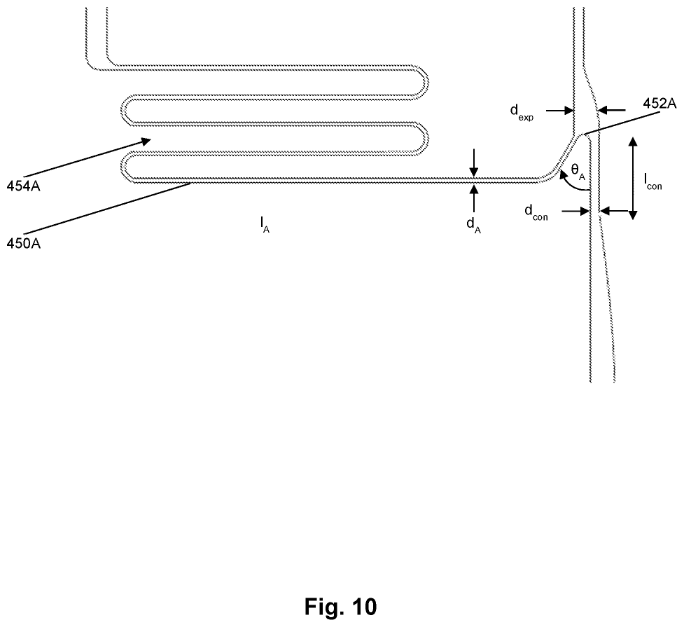

FIG. 10 schematically depicts the microfluidic device 400 of FIG. 8, in more detail. Particularly, FIG. 10 shows an enlarged portion of region B of FIG. 9.

Particularly, the microfluidic device 400 is for separating a liquid L into first and second liquid components L₁, L₂ thereof. The microfluidic device 400 comprises an inlet 430 for receiving the liquid therethrough. The microfluidic device 400 comprises a first outlet 410 for the first liquid component L₁, wherein the first outlet 410 is fluidically coupled to the inlet 430 via a first passageway 440A. The microfluidic device 400 comprises a second outlet 420 for the second liquid component L₂, wherein the second outlet 420 is fluidically coupled to the first passageway 440A via a first set of N conduits 450 (450A, 450B, 450C, 450D, 450E), wherein N is a positive integer greater than 1, wherein respective conduits 450A, 450B, 450C, 450D, 450E of the first set of N conduits 450 divide from the first passageway 440A at respective divisions 452 (452A, 452B, 452C, 452D, 452E) from the inlet 430 therealong. The respective conduits 450A, 450B, 450C, 450D, 450E of the first set of N conduits 450 are arranged to, at least in part, equalize flowrate ratios at the respective divisions 452 (452A, 452B, 452C, 452D, 452E).

Generally, the microfluidic device 400 is as described with respect to the microfluidic device 200.

The microfluidic device 400 is for separating whole blood. Preferably, the microfluidic device 400 is for separating diluted whole blood, for example whole blood diluted by a ratio of at most 10:1, preferably at most 5:1 more preferably at most 4:1, most preferably at most 1:1.

The first set of N conduits 450 consists of 5 conduits (i.e. N=5). The first conduit 450A is divided from the first passageway 440A at a spacing s_{A} from the inlet 430 therealong, thereby providing the first division 452A, specifically a bifurcation 452A. Similarly, the second conduit 450B is divided the first passageway 440A at a spacing s_{B} from the inlet 430 therealong, thereby providing the second division 452B, specifically a bifurcation 452B. Similarly, the third conduit 450C is divided from the first passageway 440A at a spacing s_{C} from the inlet 430 therealong, thereby providing the third division 452C, specifically a bifurcation 452C. Similarly, the fourth conduit 450D is divided from the first passageway 440A at a spacing s_{D} from the inlet 430 therealong, thereby providing the fourth division 452D, specifically a bifurcation 452D.

Similarly, the fifth conduit 450E is divided from the first passageway 440A at a spacing s_{E} from the inlet 430 therealong, thereby providing the fifth division 452E, specifically a bifurcation 452E.

In this example, the respective conduits 450A, 450B, 450C, 450D, 450E of the first set of N conduits 450 have respective lengths l_{A}, l_{B}, l_{C}, l_{D}, l_{E} arranged to attenuate respective flowrates therethrough according to, at least in part, the respective liquid L pressure at the respective divisions 452 (452A, 452B, 452C, 452D, 452E) 452 (452A, 452B, 452C, 452D, 452E). In this example, the length l_{A}>the length l_{B}>the length l_{C}>the length l_{D}>the length l_{E}. In this example, the length l_{A} is 6.75 mm, the length l_{B} is 5.4 mm the length l_{C} is 3.57 mm, the length l_{D} is 2.16 mm and the length l_{E} is 1.71 mm. In this example, the respective conduits 450A, 450B, 450C, 450D, 450E have the same constant rectangular cross-sectional areas, having respective equal widths d_{A}, d_{B}, d_{C}, d_{D}, d_{E} of 15 μm and respective equal heights of 20 μm.

In this example, the first passageway 440A curves from the inlet towards the first outlet along a first part of a length thereof, wherein a first radius of curvature R1 of the first part is 10 mm. In this example, the first passageway 440A tapers along the first part of the length, from a width of 470 μm to a width of 100 μm over the first part of the length, wherein the first part of the length has a length of 15 mm. In this example, the first passageway 440A curves from the inlet towards the first outlet along a second part of a length thereof, wherein a second radius of curvature R2 of the second part is 1.3 mm. In this example, the first passageway 440A has a constant cross-sectional area along the second part of the length, having a constant width of 100 μm. In this example, the first passageway 440A is linear along a third part of a length thereof, between the first part of the length and the second part of the length, wherein the first passageway 440A has a constant cross-sectional area along the third part of the length, having a constant width of 100 μm. In this example, the first passageway 440A is arranged to surround, at least in part, the first set of N conduits 450.

In this example, the first passageway 440A comprises a set of constriction members 442 (442A, 442B, 442C, 442D, 442E), wherein respective constriction members 442A, 442B, 442C, 442D, 442E of the set of constriction members 442 correspond with the respective conduits 450A, 450B, 450C, 450D, 450E of the first set of N conduits 450. The respective constriction members 442A, 442B, 442C, 442D, 442E are arranged upstream (i.e. with respect to flow of the liquid L) of the respective conduits 450A, 450B, 450C, 450D, 450E. The respective constriction members 442A, 442B, 442C, 442D, 442E are arranged proximal to, and upstream of, the respective divisions 452A, 452B, 452C, 452D, 452E. In this example, the respective constriction members 442A, 442B, 442C, 442D, 442E of the set of constriction members 442 are similar. In this example, the respective constriction members 442A, 442B, 442C, 442D, 442E of the set of constriction members 442 have the same constant rectangular cross-sectional areas, having respective equal widths d_{con} of 38 μm, heights of 20 μm and lengths of 0.302 mm.

In this example, the first passageway 440A comprises a set of expansion members 444 (444A, 444B, 444C, 444D, 444E), wherein respective expansion members 444A, 444B, 444C, 444D, 444E of the set of expansion members 440A correspond with the respective conduits 450A, 450B, 450C, 450D, 450E of the first set of N conduits 450. The respective expansion members 444A, 444B, 444C, 444D, 444E are arranged downstream (i.e. with respect to flow of the liquid L) of the respective conduits 450A, 450B, 450C, 450D, 450E. The respective expansion members 444A, 444B, 444C, 444D, 444E are arranged proximal to, and downstream of, the respective divisions 452A, 452B, 452C, 452D, 452E. In this example, the respective expansion members 444A, 444B, 444C, 444D, 444E of the set of expansion members 440A are similar. In this example, the respective expansion members 444A, 444B, 444C, 444D, 444E of the set of expansion members 440A have the same non-constant rectangular cross-sectional areas, having widths that enlarge smoothly and arcuately away from the respective divisions 452A, 452B, 452C, 452D, 452E to respective maximum widths dₑₓₚ and reduce smoothly and arcuately thereafter towards the respective constriction members 442A, 442B, 442C, 442D, 442E.

In this example, the conduit 450A of the first set of N conduits 450 is arranged at an acute angle θ_{A} to the first passageway 440. In this example, the acute angle θ_{A} is approximately 45°. The respective conduit 450B, 450C, 450D, 450E of the first set of N conduits 450 are arranged similarly at respective acute angles θ_{B}, θ_{C}, θ_{D}, θ_{E} to the first passageway 440, in which the angle θ_{A}=θ_{C}=θ_{D}=θ_{D}=θ_{E}.

In this example, the first passageway 440A defines a stepped linear flow path of the liquid via the respective divisions, wherein a wall, for example adjacent to the first set of N conduits 450, of the first passageway 440A extending between the respective divisions 452 is stepped. Particularly, the wall, adjacent to the first set of N conduits 450, of the first passageway 440A extending between the respective divisions 452 comprises linear parts through the constriction members 442 (442A, 442B, 442C, 442D, 442E) and through the expansion members 444 (444A, 444B, 444C, 444D, 444E) therebetween, in which the successive linear parts are stepped at the respect divisions. In contrast, an opposed wall, opposed to the first set of N conduits 450, of the first passageway 440A extending between the respective divisions 452 comprises alternating linear parts through the constriction members 442 (442A, 442B, 442C, 442D, 442E) and smoothly curved parts through the expansion members 444 (444A, 444B, 444C, 444D, 444E) therebetween, in which the smoothly curved parts are similar.

In this example, the conduit 450A of the first set of N conduits 450 is arranged boustrophedonically, having three parallel legs 454A of equal lengths b_{A}. In this example, the conduit 450B of the first set of N conduits 450 is arranged boustrophedonically, having three parallel legs 454B of equal lengths b_{B}. In this example, the conduit 450C of the first set of N conduits 450 is arranged boustrophedonically, having three parallel legs 454C of equal lengths b_{C}. In this example, the length b_{A}>the length b_{B}>the length b_{C}. In this example, the conduits 450D, 450E of the first set of N conduits 450 are not arranged boustrophedonically.

In this example, the inlet 430, the first outlet 410 and the second outlet 420 are arranged collinearly, thereby defining an axis Y.

In this example, the first outlet 410 is fluidically coupled to the inlet 430 via a second passageway 440B. In this example, the second 420 outlet is fluidically coupled to the second passageway 440B via a second set of M conduits 450 (450F, 450G, 450H, 450I, 450J), wherein M is a positive integer greater than 1, wherein respective conduits of the second set of M conduits 450 (450F, 450G, 450H, 450I, 450J) divide from the second passageway 440B at respective divisions 452 (452F, 452G, 452H, 452I, 452J) from the inlet 430 therealong. In this example, the respective conduits of the second set of M conduits 450 (450F, 450G, 450H, 450I, 450J) are arranged to, at least in part, equalize flowrate ratios at the respective divisions 452 (452F, 452G, 452H, 452I, 452J).

The second passageway 440B and the second set of M conduits 450 (450F, 450G, 450H, 450I, 450J) are as described with respect to the first passageway 440A and the first set of N conduits 450 (450A, 450B, 450C, 450D, 450E), respectively. In this example, N and M are equal, to five.

In this example, the microfluidic device 400 comprises a first outlet passageway 460A fluidically coupled to the second outlet 420 and to the first set of N conduits 450 (450A, 450B, 450C, 450D, 450E), wherein the second outlet 420 is fluidically coupled to the first passageway 440A via the first set of N conduits 450 (450A, 450B, 450C, 450D, 450E) and the first outlet passageway 460A.

In this example, the microfluidic device 400 comprises a second outlet passageway 460B fluidically coupled to the second outlet 420 and to the second set of M conduits 450 (450F, 450G, 450H, 450I, 450J), wherein the second outlet 420 is fluidically coupled to the second passageway 440B via the second set of M conduits 450 (450F, 450G, 450H, 450I, 450J) and the second outlet passageway 460B.

In this example, the first passageway 440A and the first set of N conduits 450 (450A, 450B, 450C, 450D, 450E) are arranged symmetrically about the axis Y with respect to the second passageway 440B and the second set of M conduits 450 (450F, 450G, 450H, 450I, 450J). The second passageway 440B is a reflection of the first passageway 440A, in which the axis Y is a mirror line, thereby defining a heart-shape. The respective conduits 450F, 450G, 450H, 450I, 450J are respective reflections of the respective conduits 450A, 450B, 450C, 450D, 450E, in which the axis Y is the mirror line.

In this example, the microfluidic device 400 is provided on a rectangular lab-on-a-chip device 40, having four (4) apertures 41A, 41B, 41C, 41D therethrough provided at corners thereof for securing in use. Alternative methods of securing in use are known.

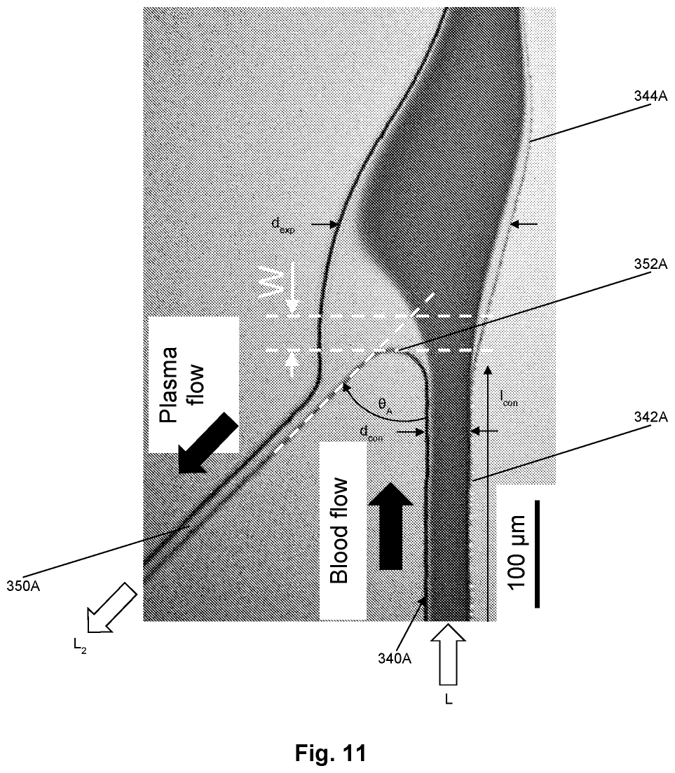

FIG. 11 schematically depicts the microfluidic device 300 of FIG. 5, in more detail, in use. Particularly, FIG. 11 is a photograph of the microfluidic device 300 showing the division 352A during separation of plasma (i.e. the second liquid component L₂) from blood (i.e the liquid L). A width W of a cell-free layer at the division 352A is approximately 30 μm.

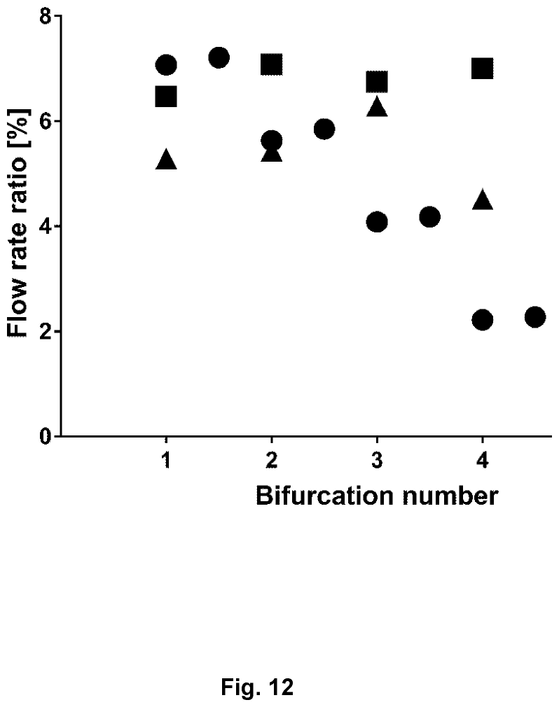

FIG. 12 is a graph showing results for microfluidic devices according to exemplary embodiments compared with a conventional microfluidic device. Particularly, the graph shows flow rate ratios between a first passageway and successive conduits of a first set of N conduits at each division, obtained from Computational Fluid Dynamics (CFD) simulations, for microfluidic devices according to the exemplary embodiments (squares and triangles) compared with the conventional microfluidic device (circles). For the microfluidic devices according to the exemplary embodiments (squares and triangles), 4 bifurcations were provided on one side of the first passageway and thus the bifurcation numbers are 1, 2, 3 and 4. For the conventional microfluidic device (circles), 8 bifurcations were staggered on alternate sides, including at intermediate spacings and thus the bifurcation numbers are 1, 1.5, 2, 2.5, 3, 3.5, 4 and 4.5. The first set of N conduits of the conventional microfluidic device are not arranged to, at least in part, equalize flowrate ratios at the respective divisions, having equal respective fluidic resistances provided by the respective conduits having equal respective lengths, cross-sectional areas and cross-sectional shapes. The flow rate ratios for the microfluidic devices according to the exemplary embodiments (squares and triangles) are relatively more constant than for the conventional microfluidic device (circles).

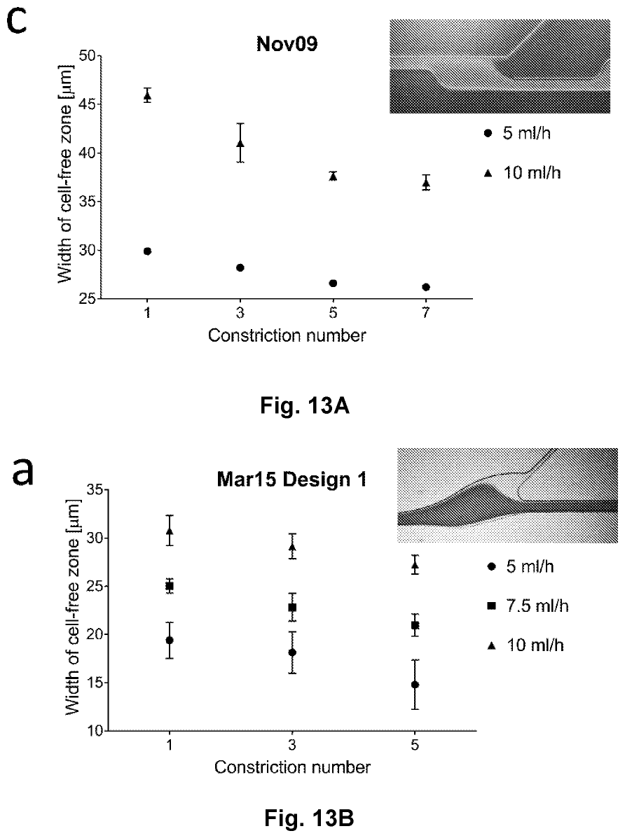

FIG. 13A is a graph showing results for a conventional microfluidic device and FIG. 13B is a graph showing results for a microfluidic device according to exemplary embodiment. Particularly, the graphs show the effects of input flow rates (circles: 5 ml/h; triangles: 10 ml/h) on widths of cell-free zones at respective divisions (labelled as constriction number) for separation of blood (diluted 1:1). Insets show photographs of the first division of each of the microfluidic devices at input flow rates 10 ml/h. Error bars denote SD, n=3, except single data points for the conventional microfluidic device at 5 ml/h. For the microfluidic device according to the exemplary embodiment, the indicated flow rate values denote branch flow rates, inlet flow rates are two times higher. For the conventional fluidic device, a width of the cell-free layer is within approximatively 20% of the maximum cell-free zones (FIG. 13A). In contrast, for the microfluidic device according to the exemplary embodiment, the width of the cell-free layer is reduced to within approximatively 10% of the maximum cell-free zones (FIG. 13B).

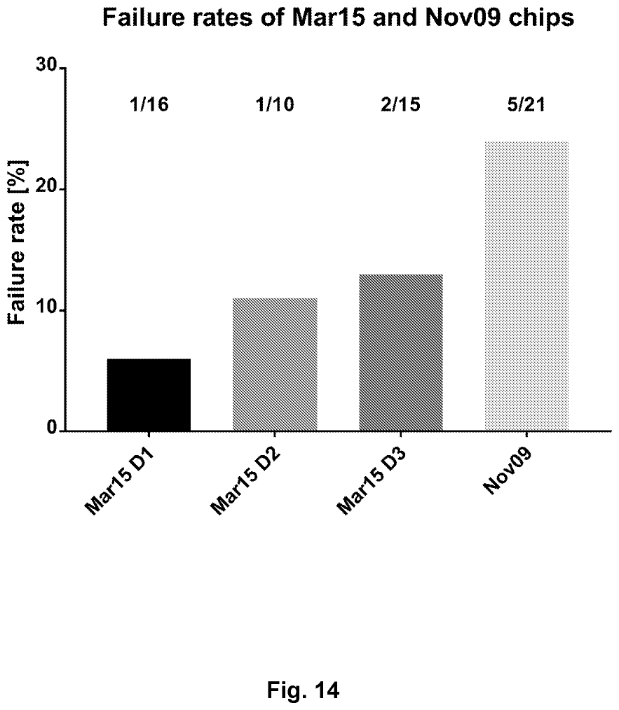

FIG. 14 is a graph showing results for microfluidic devices according to exemplary embodiments compared with a conventional microfluidic device. Particularly, the graph shows failure rates of the microfluidic devices according to the exemplary embodiments (labelled as Mar15 D1, Mar15 D2 and Mar15 D3) compared with the conventional microfluidic device (labelled as Nov09). Failure is defined as a pump stall event prior to emptying a 3 mL syringe through the respective microfluidic devices. A ratio of failed separations to all separations is indicated above the columns. The data are compiled from various experiments for blood. Dilution was 1:1 for Mar15 D1 & D3 and Nov09, while dilutions were from 1:3 to 1:10 for Mar15 D2. Inlet flow rates ranged from 10 to 20 ml/h. The lower failure rates may be at least partly attributed to the respective intersections of the respective conduits and the first passageway at the respective divisions defining arcuate flow paths of the second liquid component, as described previously.

Although a preferred embodiment has been shown and described, it will be appreciated by those skilled in the art that various changes and modifications might be made without departing from the scope of the invention, as defined in the appended claims and as described above.

In summary, the invention provides a microfluidic device for separating a liquid into first and second liquid components thereof at an improved throughput, such as at a higher flowrate and/or a higher pressure of the liquid, at improved yields and/or purities of the separated first and second liquid components. The microfluidic device is suitable for inline processing, such as on lab-on-a-chip devices. The microfluidic device is suitable for separating biological fluids, for example whole blood into plasma and waste.

Attention is directed to all papers and documents which are filed concurrently with or previous to this specification in connection with this application and which are open to public inspection with this specification, and the contents of all such papers and documents are incorporated herein by reference.

All of the features disclosed in this specification (including any accompanying claims and drawings), and/or all of the steps of any method or process so disclosed, may be combined in any combination, except combinations where at most some of such features and/or steps are mutually exclusive.

Each feature disclosed in this specification (including any accompanying claims, and drawings) may be replaced by alternative features serving the same, equivalent or similar purpose, unless expressly stated otherwise. Thus, unless expressly stated otherwise, each feature disclosed is one example only of a generic series of equivalent or similar features.

The invention is not restricted to the details of the foregoing embodiment(s). The invention extends to any novel one, or any novel combination, of the features disclosed in this specification (including any accompanying claims and drawings), or to any novel one, or any novel combination, of the steps of any method or process so disclosed.

1-20. (canceled)

21. A microfluidic device for separating a liquid into first and second liquid components thereof, the microfluidic device comprising:
an inlet for receiving the liquid therethrough;
a first outlet for the first liquid component, wherein the first outlet is fluidically coupled to the inlet via a first passageway; and
a second outlet for the second liquid component, wherein the second outlet is fluidically coupled to the first passageway via a first set of N conduits, wherein N is a positive integer greater than 1, wherein respective conduits of the first set of N conduits divide from the first passageway at respective divisions from the inlet there-along,
wherein:
the respective conduits of the first set of N conduits are arranged to, at least in part, equalize flowrate ratios at the respective divisions;
the first passageway comprises a set of expansion members; and
respective expansion members of the set of expansion members correspond with the respective conduits of the first set of N conduits.

22. The microfluidic device according to claim 21, wherein the respective conduits of the first set of N conduits are arranged to, at least in part, equalize flowrate ratios at the respective divisions by having respective fluidic resistances arranged to attenuate respective flowrates of the second liquid component therethrough.

23. The microfluidic device according to claim 22, wherein the respective fluidic resistances are provided, at least in part, by the respective conduits having respective lengths, cross-sectional areas, cross-sectional shapes and/or internal surfaces arranged to attenuate respective flowrates of the second liquid component therethrough according to, at least in part, respective liquid pressures at the respective divisions.

24. The microfluidic device according to claim 23, wherein the respective fluidic resistances are provided, at least in part, by the respective conduits having respective lengths arranged to attenuate respective flowrates of the second liquid component therethrough according to, at least in part, respective liquid pressures at the respective divisions.

25. The microfluidic device according to claim 21, wherein the first passageway tapers from the inlet towards the first outlet along at least a part of a length thereof.

26. The microfluidic device according to claim 21, wherein either:
the first passageway curves from the inlet towards the first outlet along a first part of a length thereof, or
the first passageway tapers along the first part of the length.

27. The microfluidic device according to claim 21, wherein either:
the first passageway curves from the inlet towards the first outlet along a second part of a length thereof, or
the first passageway has a constant cross-sectional area along the second part of the length.

28. The microfluidic device according to claim 21, wherein the first passageway defines a linear or a non-linear flow path of the liquid via the respective divisions.

29. The microfluidic device according to claim 21, wherein:
the respective conduits divide from the first passageway at the respective divisions by being arranged at respective acute angles thereto, and
respective intersections of the respective conduits and the first passageway at the respective divisions define arcuate flow paths of the second liquid component.

30. The microfluidic device according claim 21, wherein the first passageway comprises a set of constriction members, wherein respective constriction members of the set of constriction members correspond with the respective conduits of the first set of N conduits.

31. The microfluidic device according to claim 21, wherein a conduit of the first set of N conduits is arranged boustrophedonically, having parallel legs of equal lengths.

32. The microfluidic device according to claim 21, wherein the microfluidic device is arranged to reduce or avoid dead volumes.

33. The microfluidic device according to claim 21, wherein:
the first outlet is fluidically coupled to the inlet via a second passageway;
the second outlet is fluidically coupled to the second passageway via a second set of M conduits, M being a positive integer greater than 1,
respective conduits of the second set of M conduits divide from the second passageway at respective divisions from the inlet there-along; and
the respective conduits of the second set of M conduits are arranged to, at least in part, equalize flowrate ratios at the respective divisions.

34. The microfluidic device according to claim 21, wherein:
the microfluidic device is a blood separation device, and
the second liquid component comprises separated plasma.

35. A lab-on-a-chip device comprising a microfluidic device according to claim 21.

36. A method of separating blood using a microfluidic device according to claim 21, the method comprising:
pumping or injecting the blood into the microfluidic device via the inlet;
separating the blood into the first liquid component and the second liquid component, wherein the second liquid component comprises and/or is separated blood plasma; and
collecting the second liquid component from the second outlet.

37. The method according to claim 36, wherein the second liquid component comprises one of:
at most 10% leukocytes, platelets and/or erythrocytes by mass,
at most 5% eukocytes, platelets and/or erythrocytes by mass,
at most 3% eukocytes, platelets and/or erythrocytes by mass,
at most 2% eukocytes, platelets and/or erythrocytes by mass,
at most 1% eukocytes, platelets and/or erythrocytes by mass,
at most 0.5% eukocytes, platelets and/or erythrocytes by mass, or
at most 0.1% leukocytes, platelets and/or erythrocytes by mass.

38. The method according to claim 36, wherein the blood is diluted whole blood diluted by a ratio of one of: at most 10:1, at most 5:1, at most 2:1, or at most 1:1.

39. The method according to claim 36, wherein the blood is whole blood.

40. The method according to claim 36, comprising pumping or injecting the blood at a flow rate in one of: a range from 1 to 100 ml/hr, a range from 2 to 50 ml/hr, or a range from 5 to 30 ml/hr.
